(19) United States
(12) Patent Application Publication
(10) Pub. No.: US 2024/0077421 A1
(43) Pub. Date: Mar. 7, 2024

(54) RAPID, NON-INVASIVE AGE DETERMINATION OF PLASTICS

(71) Applicant: Regents of the University of Minnesota, Minneapolis, MN (US)

(72) Inventors: Margaret Marie Elmer-Dixon, Appleton, WI (US); Brian Hinderliter, Duluth, MN (US); Melissa Ann Maurer-Jones, Duluth, MN (US); Jonathan Michael Tigner, Apple Valley, MN (US)

(21) Appl. No.: 18/455,346

(22) Filed: Aug. 24, 2023

Related U.S. Application Data
(60) Provisional application No. 63/494,974, filed on Apr. 7, 2023.
(60) Provisional application No. 63/373,399, filed on Aug. 24, 2022.

(51) Int. Cl. G01N 21/64; G01N 33/44

(52) U.S. Cl. CPC G01N 21/6428; G01N 21/6456; G01N 33/442; G01N 2021/6439

(57) ABSTRACT

In general, technical aspects of the current disclosure include determining an age of a plastic material. In one example, a method may include controlling a light source to deliver first light to a plastic material stained with a stain; receiving, by a light detector, second light, the second light comprising at least some of the first light that has interacted with the plastic material stained with the stain; generating, by the light detector, light information representative of the second light; determining, by processing circuitry and based on the light information, an age of the plastic material; and outputting, by the processing circuitry and for display at a display of a computing device, the age of the plastic material.

This disclosure claims the benefit of, and priority to, U.S. Provisional Patent Application No. 63/373,399, entitled “RAPID, NON-INVASIVE AGE DETERMINATION OF PLASTICS” and filed Aug. 24, 2022, and U.S. Provisional Patent Application No. 63/494,974, entitled “RAPID, NON-INVASIVE AGE DETERMINATION OF PLASTICS,” filed Apr. 7, 2023, the entire contents of application nos. 63/373,399 and 63/494,974 being incorporated herein.

### TECHNICAL FIELD

The disclosure relates to spectral imaging.

### BACKGROUND

Using light to interact with matter can be used to discover physical properties of the matter. For example, a laser light can be shined on matter and the laser light that interacts with the matter can be received by an image sensor. The received light may be processed to determine physical characteristic of the matter, for example, by comparing physical properties of the received light with theoretical or previous experimental results.

### SUMMARY

This disclosure includes systems, devices, and methods related to determining an age of a plastic material using light and a stain. In one example, a method may include controlling a light source to deliver first light to a plastic material stained with a stain. The system can then receive, by a light detector, second light that is caused by the first light from the light source that has interacted with the plastic material stained with the stain. The system can then generate, by the light detector, light information representative of the second light and determine, by processing circuitry and based on the light information, an age of the plastic material. In some examples, the system may output, for display at a display of a computing device, the age of the plastic material. In this manner, the system may be configured to determine an age of the plastic material and, in some examples, sort plastic samples based at least in part of the age of each plastic sample.

In one example, a system configured to perform a method including controlling a light source to deliver first light to a plastic material stained with a stain; receiving, by a light detector, second light, the second light comprising at least some of the first light that has interacted with the plastic material stained with the stain; generating, by the light detector, light information representative of the second light; determining, by processing circuitry and based on the light information, an age of the plastic material; and outputting, by the processing circuitry and for display at a display of a computing device, the age of the plastic material.

In one example, a computing device including a display; and processing circuitry having access to a memory device, the processing circuitry configured to: generate light information representative of fluorescent light emitted from a combination of Nile red stain and a plastic material; determine, based on the light information, an age of the plastic material from the processing; and control the display to output the determined age of plastic material.

In one example, a system including a light source configured to direct light on a combination of plastic material and Nile red stain; and a computing device including processing circuitry having access to a memory device, the processing circuitry configured to: generate fluorescent light emitted from the solution of the Nile red and plastic material; process the received fluorescent light; determine an age of the plastic material from the processing; an output the determined age of plastic material and the processed data within a display of the computing device.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual diagram illustrating an example imaging system according to techniques described in this disclosure.

FIG. 1B is a conceptual diagram illustrating an example imaging system according to techniques described in this disclosure.

FIG. 1C is a functional block diagram illustrating example components of a system for determining an age of plastic samples as described herein.

FIG. 2 depicts fours graphs mapping example signal intensities at various wavelengths, according to techniques described herein.

FIGS. 3A and 3B depict two graphs mapping example representative collective signal intensities at various wavelengths, according to techniques of this disclosure.

FIG. 3C is a graph of example overlaid fluorescence spectra of different aged material stained with Nile red.

FIGS. 4A-4C depict three graphs of various example peak intensities of respective wavelengths as a function of irradiation time, in accordance with techniques of this disclosure.

FIGS. 5A, 5B depict two example graphs: a first graph plotting a peak ration intensity and a second graph mapping a center of mass of various wavelengths, in accordance with techniques of this disclosure.

FIGS. 6A, 6B depict two example graphs: a first graph plotting a peak ration intensity and a second graph mapping a center of mass of various wavelengths, in accordance with techniques of this disclosure.

FIGS. 7A, 7B depict two example graphs: a first graph plotting a peak intensity ratio and a second graph mapping a center of mass of various wavelengths, in accordance with techniques of this disclosure.

FIG. 8 is a graph of example fluorescence spectra at different ages using Nile red.

FIGS. 9A-9D are graphs of fluorescence spectra of different aged plastic samples.

FIG. 10 depicts an example diagram of a flowchart for an operation of determining an age of a plastic material, according to techniques described in this disclosure.

FIG. 11A, 11B are graphs of example fluorescence spectra of for materials stained with Nile red for different durations.

FIGS. 12A-12I are graphs of example fluorescence spectra of for photo-degraded thin files stained with Nile red for different durations.

FIG. 13 includes graphs of fluorescence peak intensities at different wavelengths over time for polyethylene (PE) samples.

FIG. 14A, 14B are graphs of example carbonyl index value changes with irradiation time for different thickness samples.

FIG. 15A, 15B are graphs of example contact angle measurement changes with irradiation time for different thickness samples.

FIG. 16A, 16B are graphs of example bulk crystallinity changes with irradiation time for different thickness samples.

FIG. 17A, 17B are graphs of example peak fluorescence intensity ratios and center of masses compared with bulk crystallinity across film irradiation for different thickness samples.

FIG. 18 is a graph of an example melting curve of 30 um polyethylene as measured by differential scanning calorimetry.

FIGS. 19A, 19B are graphs of example peak fluorescence intensity ratio changes compared with contact angle measurements across film irradiation of different thickness samples.

FIGS. 20A, 20B are graphs of example center of mass changes compared with contact angle measurements across film irradiation of different thickness samples.

FIGS. 21A-21F are graphs of example fluorescence spectra of thermally degraded PE thin films stained with Nile red.

FIGS. 22A, 22B are graphs of example peak fluorescence intensity ratios (A) and center of mass (B) changes with film irradiation for thermally degraded PE samples.

FIGS. 23A, 23B, and 23C are graphs of example peak fluorescence intensity ratio changes of photo-degraded (blue) (A) and thermally degraded (red) (B) compared with carbonyl index of PE with overlaid data for visual comparison of trends (C).

FIGS. 24A, 24B are graphs of example peak fluorescence intensity ratio (A) and center of mass (B) changes with film irradiation for photo-degraded PP thin film samples.

FIGS. 25A, 25B are graphs of example fluorescence Spectra of stained poly-L-lactic acid films at different excitation wavelengths.

FIGS. 26A-26D are graphs of example FTIR absorbance measurements of unaged commercial plastic samples.

FIGS. 27A, 27B are graphs of example peak fluorescence intensity ratio (A) and center of mass changes (B) with film irradiation time for different commercial plastic samples.

FIGS. 28A, 28B are graphs of example raw fluorescence signals of unirradiated (A) and irradiated (B) plastic samples.

FIG. 29A is a graph of average fluorescence intensity for different wavelengths.

FIG. 29B is a graph of average center of mass and average carbonyl index for different film ages.

FIG. 30 is a graph of average center of mass over different film ages.

FIG. 31 is a graph of perceived wavelength over different ratios of red to green from RGB data.

FIG. 32A is a conceptual diagram of an example technique for obtaining image data for a sample of plastic subject to a specific dye.

FIG. 32B is a flow chart of an example technique for determining age of samples from obtained image data.

Like reference characters denote like elements throughout the figures and text.

### DETAILED DESCRIPTION

This discloses describes devices, systems, and techniques related to determining an age or degradation of a polymer material. While plastics have been incredibly useful in developing the quality of life for people all around the globe, they have a rather substantial impact on the natural environment. From the 1950's to 2015, plastic production increased approximately 200 times. It is estimated that of the 8.3 billion tons (Bt) of plastics produced from 1950 to 2015 only about 7.2% has been recycled and 9.6% incinerated. This means that nearly 83% of all plastic produced during that time became waste after use. Around 5.7 Bt (˜69%) of this plastic was discarded as waste, with 4.9 Bt (˜86% of discarded waste) placed into landfills or directly into the natural environment through improper waste management.

The challenge to address plastic waste becomes increasingly important as production is consistently on the rise. In particular, plastic recycling is becoming an ever more pressing issue as petrochemical feedstocks begin to deplete. Plastic recycling reduces our current reliance on fossil fuels and helps reduce the massive amounts of plastic waste. However, compared to other currently used recyclable materials (i.e., glass, paper, or metal), recycling and recovery rates for plastic are rather low. In part, the huge diversity of plastics decreases recycling efficiency unlike that of paper, glass or metal. Even in countries with high recovery of plastics as waste, the rate of recycling can be very low, where plastics are used for energy recovery through incineration. An aspect of plastic recycling is its dependence on the quality of the polymer material being recycled. Plastics undergo photochemical and thermal degradation that changes the surface hydrophobicity, chemical composition, surface and bulk crystallinity, and mechanical strengths. Degraded plastic cannot be recovered mechanically or chemically because they do not share the same chemical and physical properties with new plastics and the recycling of them would yield lower quality and potentially flawed materials. In addition, plastic is difficult and time consuming to characterize for sorting or any other purpose.

A current problem in the art with direct image-to-age reporting processing is that light, when interacting with matter, can undergo six major processes, each with a distinct outcome that results in perceived light detected by the naked eye. Further, the resultant light from these processes can undergo color mixing further complicating exactly what it means when a perceived color is detected. This essentially means that it is very difficult to convert a computer color code red-green-blue (RGB) to a specific wavelength of light. There is no standard method in the art for doing this.

One solution to minimizing plastic pollution is to improve reuse and recycling strategies. Yet, recycling is limited by plastic degradation. Photochemical or thermal driving forces cause the incorporation of oxygen into the backbone and chain cleavage; yet, current techniques for monitoring this plastic degradation fail to observe early stages of degradation, which is key for optimizing reusability. This disclosure is related to developing a cheap, reproducible, and nondestructive technique for monitoring degradation of polymers such as polyethylene and polypropylene materials using Nile red as one example of a fluorescent probe. Changes in Nile red's fluorescence spectra were observed upon exposure to stained, aged polyethylene and polypropylene samples. As the surface hydrophobicity of the plastic decreases, Nile red's fluorescence signal undergoes corresponding signal shift to longer wavelengths (lower energy). The trends seen in the fluorescent profile were related to more commonly used measurements of plastic degradation, namely carbonyl index from infrared spectroscopy and bulk crystallinity from calorimetry. Results demonstrate clear trends in fluorescence spectra shifts as related to the chemical and physical changes to the plastics, with trends dependent on polymer type but independent of polymer film thickness. The strength of this example technique is divided into two defined fits of the fluorescence signal; one fit characterizes the degradation throughout the whole range of degradative oxidation and the other is tailored to provide insight into the early stages of degradation. Overall, as described herein, techniques can provide a characterization tool that assesses the extent of plastics' degradation, which may ultimately impact our ability to recover plastics and minimize plastic waste.

There are many different techniques to track plastic aging. Methods monitor degrading surface properties because changes are most prominent at the surface, particularly at the beginning of aging. These surface measurements include monitoring chemistry changes by quantifying carbonyl index measurements using Fourier transform infrared spectroscopy (FTIR) and hydrophobicity with contact angle measurements. To account for the incorporation of oxygen into the backbone of the polymers, particularly polyolefins, carbonyl index as measured by IR spectroscopy has established itself as a key marker to monitor for polymer degradation.

The carbonyl index is the ratio of absorbance values of carbonyl peaks to reference areas in a plastic's IR absorption spectrum. There are ways to calculate a carbonyl index, which includes some variability of the carbonyl and reference spectral characteristics that depend on the polymer backbone and the discretion of the researcher. While using a carbonyl index can provide information about the changing plastic surface chemistry, there are a number of caveats to the technique making implementation and reproduction difficult. Carbonyl index is a measure of the chemistry on the surface, but does not provide information about the resultant changes in surface properties. In contrast, contact angle measurement is a method that allows for the monitoring of changing surface properties by monitoring the angle of contact between a materials surface and a liquid droplet. As the surface hydrophobicity changes with polymer aging and oxygen incorporation, the liquid droplet will change how it interacts with the plastics' surface. This technique can use different types of liquids in order to gain additional information about surface properties. Water can be used to test a change in hydrophobicity as the surface of a plastic changes. This technique can be useful in monitoring and understanding the changes in surface hydrophobicity of materials as their chemical composition changes. Unfortunately, static contact angle goniometry is a relatively challenging technique, particularly for morphology of plastic that is not easily flat, and is still widely in development in terms of best application and variation in measurement techniques.

Together, carbonyl index and contact angle can be used to gain a more complete understanding of surface chemical and property changes throughout aging. Both of these techniques, however, are limited in their applicability to degradation characterization. This is particularly true in their ability to characterize degradation at the early stages. Therefore, it may be helpful to employ a technique of monitoring the processes of plastic degradation that covers the range of early to late stages, to maximize its reusability and recyclability.

Fluorescence staining can be used a strategy for plastics detection. Primarily, it can be used as a detection tool for identifying plastic materials amongst a variety of substances in single waste streams or particles (i.e., micro- and nano-plastics) within natural samples. One of the prominent fluorophores that can be used is Nile red, which is arguably the most used in the microplastic community to detect and quantify microplastics. Yet, many of these fluorescent studies only collect bulk fluorescence signal at a specific emission wavelength and largely ignore important spectral information from the stain adsorbed onto plastic.

Herein, the disclosure provides a system and/or device and new analysis method for determining plastic aging, such as analysis of Nile red fluorescence that enables quantification of plastic aging. This example has various advantages, such as overcoming the limitations of other techniques like FTIR or contact angle. In some examples, this disclosure describes using the stain in a unique way and establishes a robust fluorescence method to monitor samples at various stages of photo- or thermal-aging, with materials of differing polymers and thicknesses. Aspects of the present disclosure include, for example, a technical design as well as a method for data analysis that work in concert to easily, rapidly, and non-invasively age polymers (e.g., a plastic material). The techniques may then determine how to identify aged and/or degraded polymers. Overall, these techniques may provide an inexpensive, reproducible method for quantifying changes in plastic surface properties using a well-established fluorescence probe.

In one example, the technical design of a system or device may include a laser light (e.g., a 488 nm laser light, but other wavelengths may be used, such as at least in the range of 470-490 nm), using a dispersion lens, directed at a plastic sample. The plastic may be stained (e.g., for a predetermined amount of time, such as hours, days, and so on) with Nile red (such as a commercially available, lipophilic reporter fluorophore). The 488 nm light is absorbed by the Nile red-stained plastic, resulting in a fluorescent radiation, which may depend on the local environment of the chemical. Specifically, because Nile red reports on hydrophobicity, fluorescent radiation changes from green to yellow to red as its local environment becomes more hydrophilic (it does not report the environment of water). The resultant fluoresced light passes through a green fluorescent protein (GFP) dichroic that permits passage of wavelengths ranging from 505 nm to 800 nm. The GFP dichroic further functions to eliminate scatter or reflection of 488 nm light (or 470 to 490 nm light) due to surface characteristics of the plastic. Images are acquired on the opposite side of this sample window using a CCD camera, or a CMOS commonly found in an iPhone. Software is under development to permit immediate characterization of images on a phone. MATLAB software is under development for image processing as well.

Aspects of the current disclosure further include a method that includes image acquisition. The image acquisition requires data processing utilizing currently unavailable techniques. Several caveats of the experimental design have been utilized to overcome these shortfalls.

Advantageously, technical aspects of the present disclosure can include deriving a link from an RGB code to a particular wavelength. All spectroscopic data corresponding to the fluorescence of Nile red can have spectral signals ranging from 505 nm to 700 nm. The average center of mass wavelength, representative of the perceived color emitted during fluorescence emission can range from 577 nm to 630 nm. Red (R) and green (G) color codes are generally representative of 532 nm and 630 nm, respectively. A database including wavelength center-of-masses from Nile red exposed to a range of oxidized polyethylene (PE) ranging from unaged to fully oxidized polyethylene were calculated. These wavelengths were then converted to RGB color codes using wavelength2color.m (which is a free m-file that converts wavelength to corresponding RGB code). Because Nile red does not emit a wavelength in the blue region of the light spectrum, the B terms are all zero and the value can be omitted.

According to experimental results, R-G ratios show a discrete, simply-connected characterization of the possible ratios of red to green that characterize all of the wavelengths that can possibly be perceived from the Nile red fluorescence. This elimination of the B color value and translation of the RGB code into an R-G ratio with no redundant values, permits the conversion of CCD camera image (reported in RGB) to a wavelength (reported in nm) that can be directly correlated with a carbonyl index. This means that the RGB code can be converted to a pixel by pixel age.

Advantageously, technical aspects of the present disclosure include a technique for overcoming complexity of converting RGB (e.g., the pixel intensities of a device display that processes the emitted fluorescent light) to a corresponding wavelength to extract aging information of a plastic material product design. Further, technical aspects of the present disclosure include program instructions, stored in a memory that, when executed by a processor, cause the processor to implement technical aspects of the current disclosure. Technical aspects of the current disclosure permits in-field, non-invasive data acquisition and analysis of plastic materials to determine extent of aging in a rapid, user-friendly manner.

Nile red may be used to identify micro polymer, for example, in solution without knowledge of the polymer age. Fourier-transform infrared spectroscopy (FTIR) and differential scanning calorimetry (DSC) are used to characterize and determine aging of the material. This method extends the use of Nile red by utilizing its intrinsic quantum properties to inform a user on polymer age in a non-destructive manner. FTIR requires samples to be collected and brought into the lab for testing. DSC destroys samples in the testing process and sample sizes are limited to less than 1 milliliter (mL).

However, using fluorescence to determine the age of a plastic material, as with the technical aspects of the present disclosure, does not destroy samples, can be done ‘in the field,’ and is affordable by comparison in terms of time and price point. Finally, acquisition and analysis of sample plastic materials is unrestrained (relative to the current state of the art) by equipment setup making characterizing larger objects possible. Technical aspects of the present disclosure provide improved insight into the extent of aging over large areas of plastic materials which is especially beneficial in understanding pre-aging due to manufacturing.

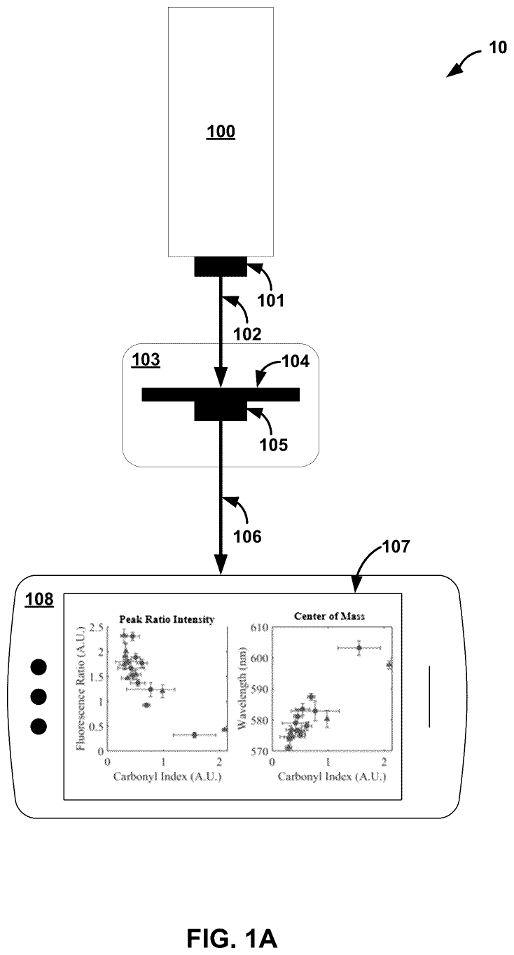

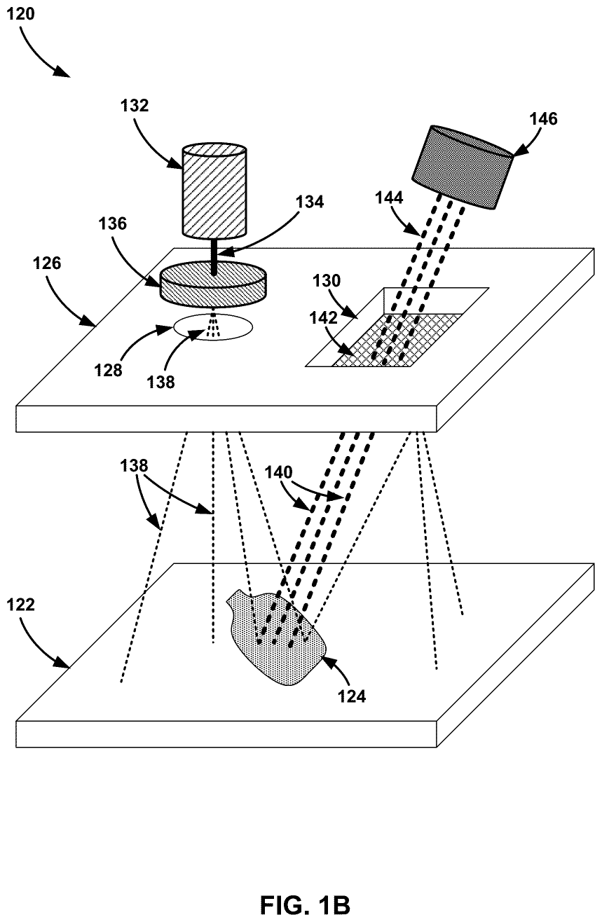

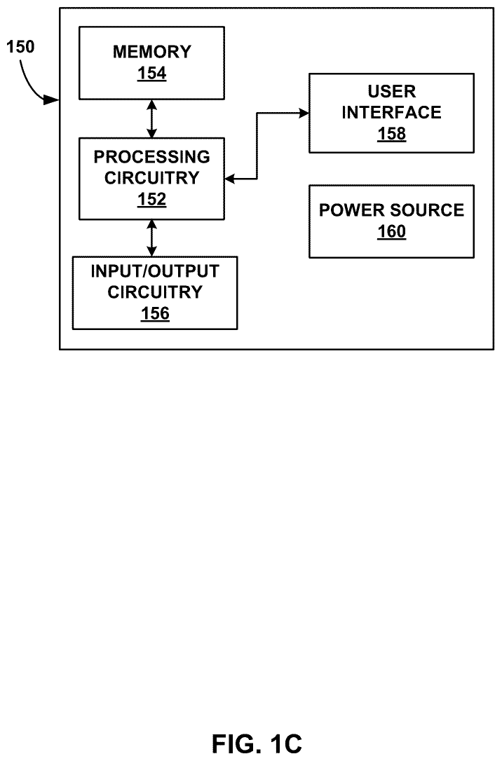

Technical aspects of the present disclosure have multiple practical improvements to the fields of academic research and industry. For example, in academic research, environmental chemists can characterize their plastic material samples in the field, rather than in a lab. A portable device, as discussed below with reference to at least FIG. 1, would also help industry field technicians who are required to extract field polymer samples for later lab testing. In industry, cable technicians characterizing power-cable lifetime could use the portable device to expedite their work, streamlining power cable characterization, saving resources (e.g., time, money, and so on) for energy companies and making estimation and determination of cable up-keep much more efficient. In the lab, materials science research could use the technical aspects of this disclosure to better characterize work. Technical aspects of this disclosure include providing information on potential degradation of plastic material that can aid in the research and development of improved degradable or non-degradable plastics. Finally, the technical aspects of this disclosure could easily be scaled such as to permit industrial characterization, such as that required in plastic material recycling, permitting recovery of previously ‘undesirable’ plastics for repurposing.

Spectroscopic data is being formulated into a publication to establish the link between Nile red fluorescence and film age to establish it as an oxidation and thus, age reporter. Preliminary microscopy work has shown that the technique works well to elucidate 200 nanometer (nm)-2 micrometer (um) structures that arise during film aging. A prototype has taken preliminary images demonstrating the functionality of the technique. This data has been used to modify the current microscopy technique. Current work on data analysis code to output images representative of perceived color change is underway. Once this is established, an iOS code will be used to implement the acquisition on a handheld device.

FIG. 1A is a block diagram illustrating an example imaging system according to techniques described in this disclosure. Generally, system 10 may include a light source configured to activate degradation reporter dyes, a dichroic window configured to remove light directly from the activation light source, and a light detector (e.g., a camera configured to capture light and generate an image output in RGB). The technique may also include the corresponding reporter dye that can be applied to samples. In this example, imaging system 10 includes a light source 100 (e.g., laser light source in some examples) and a dispersion lens 101. Laser light 100 directs a first light 102 (e.g., with a 488 nm wavelength; other wavelengths, such as 470-490 nm may be used) through dispersion lens 101 onto a plastic material 104 (e.g., any type of polymer, including polypropylene, polyethylene terephthalate, poly-lactic acid, and so on) centered on a stage 103. Plastic material may have been previously stained with a solution (e.g., Nile red, which may be commercially available in the form of a lipophilic reporter fluorophore). The Nile red may be added to the plastic in any quantity and for any amount of time (e.g., four hours, twenty-four hours, and so on) before first light 102 is directed at plastic material 104. A specific reporter dye, such as Nile red, may have been applied to plastic material 104 prior to illumination with first light 102. For example, a liquid containing the reporter dye may be sprayed onto the plastic samples. As another example, the plastic samples may be dunked into a liquid bath containing the reporter dye. Although one specific dye is described herein, the liquid may contain multiple dyes in some examples that may be specific for respective types of plastic.

First light 102 is absorbed by the Nile red, when the plastic samples are exposed to the first light, resulting in a fluorescent radiation (not shown in FIG. 1A), which may depend on the local environment of the chemical. Specifically, Nile red reports on hydrophobicity, the fluorescent radiation may change visible characteristics of the fluorescence wavelengths from green to yellow to green as the local environment of the Nile red becomes more hydrophilic. A green fluorescence protein (GFP) dichroic 105 (e.g., an example light filter) is configured to filter the fluorescent radiation to produce a second light 106. GFP dichroic 105 permits passage of fluorescent radiation having wavelengths ranging from 505 nm to 800 nm. GFP dichroic 105 further functions to reduce/eliminate scattering or reflection of fluorescent light with wavelengths centering around 488 nm, which may be caused from the structure or surface of plastic material 104. Other filters may include various dichroic mirrors or long pass filters. Example long pass filters may be configured to pass wavelengths such as 490 nm, 505 nm, 510 nm, 525 nm, 530 nm, 535 nm, 550 nm, 567 nm, 605 nm, 638 nm, 650 nm, or 730 nm.

A camera 108 (which may be a charged-coupled device (CCD) camera, complementary metal oxide semiconductor (CMOS) camera, such as found in commercially available smart devices, or any other light detection device) may be carried by a computing device and positioned to receive second light 106, such that the lens (not shown in FIG. 1A) of camera 108 is opposite to laser light 100. Optical receiving lens of camera 108 receives second light 106. Processing circuitry (not shown in FIG. 1A), which may be communicatively coupled to (for example, a computing system communicating wirelessly with the camera 108), or integrated within, camera 108, may process second light 106. For example, processing second light 106 may include converting the RGB pixel intensities from lens of camera 108 to corresponding wavelengths of fluorescent radiation. For example, lens of camera 108 may receive second light 106 and determine particular pixel intensities of the received light with a standard Bayer filter. The determined RGB pixel intensities may then be converted to a corresponding set of respective wavelengths.

Processing circuitry within camera 108, in a device that includes camera 108, or in a separate computing device (e.g., a tablet computer, notebook computer, desktop computer, remote server, etc.) may analyze the detected second light 106. Processing circuitry may execute code including instructions for processing image data, extracting degradation data from images, and determining age of plastic sample(s) in the images. Processing circuitry may use Matlab code, python code, and/or any other type of code that achieves the functions and techniques described herein. Generally, camera 180 detects the resultant fluorescence (e.g., the color emission) of the reporter dye according to the second light 106 that contacts the sensor of camera 108. For the reporter dye of Nile red, unadulterated plastics can show up green, but appear yellow and red as they age over time.

In one example, the processing circuitry may process second light 106 as follows, which includes experimental results incorporated into a database stored within, or communicatively coupled to, for example, camera 108, and may be compared to the converted wavelengths. All spectroscopic data corresponding to the fluorescent radiation of Nile red should have spectral signals ranging from 505 nm to 700 nm. The average center of mass wavelength, representative of the perceived color emitted during fluorescence may range from 577 nm to 630 nm. R and G color codes are generally representative of 532 nm and 630 nm. A database (e.g., stored within, or communicatively coupled to, camera 108) of possible wavelength center of masses from fluorescence radiation of Nile red exposed to unaged to fully oxidized polyethylene were calculated in experiments. These wavelengths were then converted to RGB color codes using wavelength2color.m, a free m file that converts wavelength to RGB code.

The center of mass of a spectrum as described herein can be determined by weighting the wavelength location (xᵢ) by the spectral signal (Sᵢ) at that location then summing over all wavelengths in the spectrum and dividing by the sum of all spectral signals in the spectrum,

$$y = \frac{{\sum }_{i} ( {S}_{i} {x}_{i} )}{{\sum }_{i} {S}_{i}} \qquad ( 1 )$$

where the resultant value, y, is the center of mass of the spectrum measured in units of wavelength.

Because Nile red fluorescence radiation does not emit a wavelength in the blue region of the spectrum, the B terms of RGB code are all zero and the value can be omitted. Ratios of R and G show a discrete, simply connected characterization of the possible ratios of red to green that characterize all of the wavelengths that can possibly be perceived from the Nile red fluorescence. This elimination of the B color value and translation of the RGB code into an R-G ratio with no redundant values, permits the conversion of CCD camera image (reported in RGB) of camera 108 to a wavelength (reported in nm) that can be directly correlated with carbonyl index. This means that the RGB code can be converted to a pixel by pixel age.

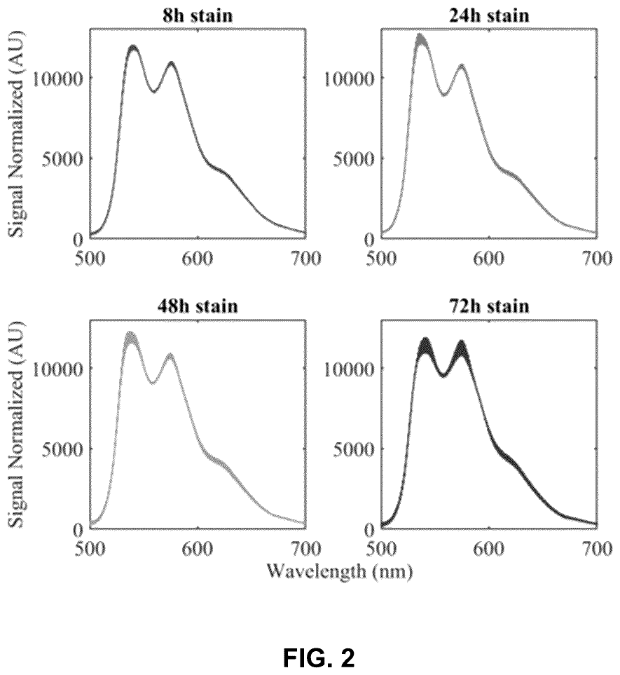

Camera 108 may output via display 107 (e.g., a display device that may be configured to present a graphical user interface) an estimated age of plastic (e.g., 10 years, 100 years, and so on) material 104, which is derived from the ratio of red to green wavelengths (and/or the ratio of green to red wavelengths) of fluorescent radiation. Processed second light 106 may be outputted in the form of charts and graphs (as shown in FIGS. 2-6B) via display 107 (e.g., a graphical user interface) of camera 108.

Example materials and methods are described below. These are only some examples, and the techniques may be used in other examples and for other samples. For the data described herein, polymer thin film samples used in this study were polyethylene (PE; 30 and 50 μm thick), polypropylene (PP; 30 μm thick), poly-L-lactic acid (PLLA; 50 μm thick), and polyethylene terephthalate (PET; 28 μm thick). These polymer samples did not contain UV-stabilizers, antioxidants, or other coloring additives. To relate ‘additive free’ samples to realistic samples, plastic bags from various convenience/department stores were procured (Cub Foods; Stillwater, MN; Target; Minneapolis, MN; Solo Cup Company; Lake Forest, IL; Aldi's Little Salad Bar; Batavia, IL). Nile red (CAS #7385-67-3) was purchased from Chem Impex Int'l Inc. (Wood dale, IL) and used for all samples used in the data collection herein.

Polymer samples were soaked in various solvents to remove unpolymerized monomers/oligomers or processing additives. These molecules were leached by soaking the polymer films for 24 h in each of the following: hexanes (CAS #110-54-3), followed by methanol (CAS #67-56-1), followed by doubly distilled water. This leaching process was not employed for single use bag samples obtained from convenience/department stores to better replicate “as-is” characterization of these commercial products.

As a mechanism to produce aged and degraded plastics quickly, polymer samples were exposed to UV-light. Beyond being the model aging mechanism for this study, photodegradation is a primary pathway for abiotic plastic degradation. To age the plastic samples at an accelerated rate, PE and PP plastic samples were placed in a Rayonet merry-go-round photochemical reactor (Southern New England Ultraviolet Co.; Branford, CT) and irradiated on both sides for various time periods with 16 Hg-vapor lamps (SNE Ultraviolet Co RMR-2537A; Bamford, CT) emitting photons centered at 254 nm light. There is an irradiation of approximately 2.5 Wm⁻² for this lamp set up, which forms markers of degradation on the polymer within 48 h that are equivalent to 3-4 weeks of aging in summer sunlight. PE was aged for 2, 4, 8, 12, 18, 24, and 48 hour timepoints; PP was aged for 6 and 12 hour timepoints. This intensity of light is not intended to directly mimic natural sunlight, but instead is used to artificially degraded the plastic samples at an accelerated rate to test the applicability of this fluorescence staining to track degradation. The products of aging in this manner are equivalent to those observed in aging with natural sunlight but occur at a much faster rate for convenience of research.

To ensure the photodegradation models did not induce special artifacts that would invalidate the technique to other modes of degradation, PE samples (30 μm thick) were degraded thermally by suspending plastics in an oven at 110° C. for 24, 48, 72, 192, 264, and 336 h. This temperature is relatively close to the melting point (˜120° C.) that allowed for accelerated aging while keeping the polymer film intact. The temperature chosen for aging is, again, not intended to directly mimic environmental factors, but is instead meant to produce model degraded plastics.

Nile red was dissolved in 100% ethanol (CAS #64-17-5) to a concentration of 0.05 mg/mL, or approximately 170 μM. This solution was prepared in glass containers and was stored in a dark room with an aluminum foil wrap to avoid degradation for exposure to light. Samples of each plastic at each timepoint of aging were fully submerged in 25 mL of 170 μM Nile red/ethanol solution and left to stain for eight hours, which was determined based on staining optimization experiments (see SI for details and results). After staining, plastic samples were removed from the Nile red solution, rinsed with deionized water to remove excess Nile red, and allowed to dry. Samples were stored in aluminum foil until fluorescence spectroscopy analysis. Primarily, fluorescence measurements were taken within two days after staining. Fluorescence measurements were also conducted on samples stored for ˜11 months after initial staining to test the shelf-stability of the staining.

Fluorescence measurements of stained plastic samples were taken on a Horiba Fluoromax-4 Spectrofluorometer (Horiba Scientific; Edison, NJ). Each stained sample was cut into about 1×3 cm dimensions and fluorescence measurements were taken on replicate polymer samples to assess the variability of staining. Samples were placed in a 3D printed solid state fluorescence cuvette to be analyzed. Excitation wavelength was 470 nm and emission spectra were collected from 490-700 nm with a 3 nm slit width for both excitation and emission. A minor signal from reflecting light was obtained between 490-500 nm and this region was removed for data analysis and visualization. Additional samples were prepared, and data was also collected at an excitation wavelength of 488 nm in order to investigate application of this technique to other fluorescence techniques that may use a laser or light emitting diode at that wavelength. Emission spectra were collected from 500-700 nm with a step size of 1 nm and excitation and emission slit widths of 3 nm.

Analysis of the fluorescence signal was performed in Matlab (R2022a) software (MathWorks; Natick, MA). All data were normalized by dividing the fluorescence signal of each wavelength by the sum of the total fluorescence signal. This maintains the weighting of each wavelength measurement as it is now representative of its contribution to the whole of the signal. These values were adjusted by an arbitrary factor of 10⁶ in order to visualize the normalized signal in an order of magnitude more familiar in fluorescence spectroscopy. This factor could be changed with no impact to the analysis. These normalized values were then used for all further analysis.

Attenuated total reflectance—Fourier transform infrared spectroscopy (ATR-FTIR) was performed on the samples to characterize the molecular structures on the polymer surface. A Nicolet iS50 (Thermo Fisher Scientific; Waltham, MA) with diamond ATR cell was used, collecting at least 3 spectra at different locations per sample. Each spectrum was collected through averaging 64 scans with a 4 cm⁻¹ resolution. Analysis of the carbonyl band in the spectra is one measure of the surface hydrophobicity commonly used in the literature. To quantify the carbonyl band, ATR-FTIR measurements of aged polyethylene samples were analyzed using Igor Pro 8.04 (WaveMetrics; Portland, OR) software, individually analyzing the spectra carbonyl absorption band area (1680-1800 cm⁻¹) and normalized to a reference band area (˜1380 cm⁻¹). This ratio generated a single value known as a carbonyl index. The analysis was conducted on each triplicate measurement for plastic samples and the mean and standard deviation between trials was calculated.

Contact angle measurements were conducted using static contact angle techniques on an Ossila contact angle goniometer (Ossila; Sheffield, England). Droplets of approximately 5 μL were placed onto the surface of plastic films using a 10 μL blunt tip syringe. These measurements were conducted in triplicate, using three locations on a single thin film adhered to a glass base to avoid an uneven surface. Analysis of the static contact angle was conducted on each replicate measurement using the LB-ADSA drop analysis plugin in ImageJ. Liquid density was set to the water preset value and drop width and height were adjusted until the model drop was congruent with that of the observed water drop on the plastic film. The final reading of the drop contact angle was then used from the software analysis.

DSC measurements were conducted using an established method for polyethylene on a TA Instruments DSC 250+ calorimeter (New Castle, DE, USA). For each sample, measurements were collected in triplicate using between 5 and 10 mg of thin film sample in Tzero pans. Samples were analyzed upon a heat-cool-heat cycle, with the temperatures ranging from 40-180° C. at ramp rates of 10° C./min. All analysis was conducted on the first heating of the samples. Heat flow measurements were automatically normalized by sample mass on collecting. The enthalpy of melting was calculated by integrating the observed melting curve and converted to percent crystallinity through comparison to a reference value for 100% crystalline PE of 293 J/C.

Linear regression modeling was conducted using the curve fitting tool in Matlab and R² values are reported as a measure of the quality of the linear fit. In order to compare two linear regressions, Real Statistics slopes test was employed. The Real Statistic slopes test is a test designed to determine if two regressions are statistically similar by comparing the variation in both slopes to confirm whether they are overlapping. The resultant p-values were reported as a measure of the probability of overlap of the regressions.

FIG. 1B is a conceptual diagram illustrating an example imaging system 120 that may be substantially similar to the functionality of system 10 of FIG. 1A. As shown in the example of FIG. 1B, base 122 may be a structure configured to retain sample plastics such as sample 124. System 120 includes platform 126 which defines transmission orifice 128 and reception window 130. Orifice may be an opening that may or may not have a filter or other material within orifice 128, but reception window 130 may include a filter 142 such as a GFP dichroic filter. System 120 also includes a laser 132, dispersion lens 136, and optical detector 146.

Laser 132 is a light source that produces light beam 134. For example, light beam 134 may be of a specific wavelength, such as 488 nm. Light beam 134 can pass through dispersion lens 136 which broadens light beam 134 into first light 138 that passes through orifice 138 and towards base 122 and sample 124. Some of first light 138 may be reflected office base 122 and back towards platform 126. Some of first light 138 strikes sample 124 that has been subject to the reporter dye, such as Nile red, and second light 140 is reflected towards reception window 130. Only second light 140 of certain wavelengths can pass through filter 142, such as a GFP filter configured to permit passage of wavelengths ranging from 505 nm to 800 nm. Those select wavelengths of light 144 are then detected by optical detector 146, which may be a camera or other device configured to convert light energy into an electrical signal (e.g., a charge coupled detector (CCD) or other device). Optical detector 146 may then transmit an image signal or image data representative of the electrical signals produced from light 144 to a computing device (e.g., a data acquisition device or other computing device) configured to analyze the image data and determine an age of the plastic of sample 124 as described herein.

FIG. 1C is a functional block diagram illustrating example components of a system 150 for determining an age of plastic samples as described herein. System 150 may be a computing device, such as camera 108 of FIG. 1A, a data acquisition device, a networked server, or any other computing device configured to analyze image data, determine an age of a plastic sample, generate information to display to a user, or any other function described herein.

System 150 includes processing circuitry 152, memory 154, input/output circuitry 156, user interface 158, and power source 160. Processor 152 controls user interface 158 and input/output circuitry 156, and stores and retrieves information and instructions to and from memory 154. Processing circuitry 152 may comprise any combination of one or more processors including one or more microprocessors, DSPs, ASICs, FPGAs, or other equivalent integrated or discrete logic circuitry. Accordingly, processor 80 may include any suitable structure, whether in hardware, software, firmware, or any combination thereof, to perform the functions ascribed herein to processing circuitry 152.

A user may interact with system 150 through user interface 158. User interface 158 includes a display (not shown), such as an LCD, LED display, or other type of screen configured to present information related to the status of other components, image data from sample plastic, graphs or data from any analysis of the image data, and/or age of one or more samples from the image data. In addition, user interface 158 may include an input mechanism to receive input from the user. The input mechanisms may include, for example, buttons, a keypad (e.g., an alphanumeric keypad), a peripheral pointing device or another input mechanism that allows the user to navigate through user interfaces presented by processor 152 of system 150 and provide input.

In some examples, at least some of the control of components of the imaging system, such as system 10 or 120, may be provided by processing circuitry 152. For example, processing circuitry 152 may control the acquisition of imaging data of various samples, the analysis of imaging data, which data or graphs to present to the user, etc.

Memory 154 may include instructions for operating user interface 158 and input/output circuitry 156, and for managing power source 160. Memory 154 may also store any image data or analysis data received by or generated by system 150. Memory 154 may include any volatile or nonvolatile memory, such as RAM, ROM, EEPROM or flash memory. Memory 154 may also include a removable memory portion that may be used to provide memory updates or increases in memory capacities.

Input/output circuitry 156 may enable system 150 to receive any information, such as image data or information from a camera that includes raw RGB data, for example. Input/output circuitry 156 may enable wired or wireless communication via any standard communication protocols.

Power source 160 is configured to deliver operating power to the components of system 150. Power source 160 may include a battery and a power generation circuit to produce the operating power. In some examples, the battery may be rechargeable to allow extended operation. Recharging may be accomplished by electrically coupling power source 160 to a cradle or plug that is connected to an alternating current (AC) outlet. In addition, recharging may be accomplished through proximal inductive interaction between an external charger and an inductive charging coil within system 150. In other examples, traditional batteries (e.g., nickel cadmium or lithium-ion batteries) may be used.

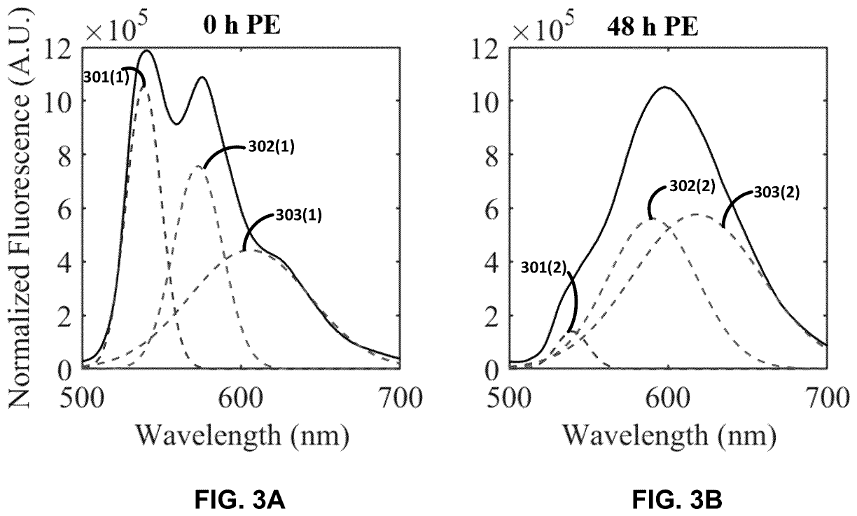

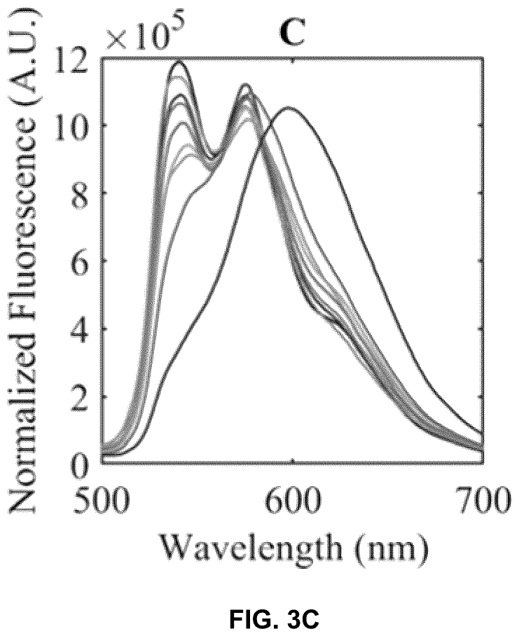

FIG. 2 depicts four plots for varying stain times of Nile red and 30 pm polyethylene film. As shown, normalized signal intensity (in arbitrary units—AU) is shown as a function of various wavelengths. For example, FIG. 2 shows four plots, with stain times of 8 hours (upper left-hand corner), 24 hours (upper right-hand corner), 48 hours (lower left-hand corner), and 72 hours (lower left-hand corner). The stain time of 72 hours provides a larger signal intensity of the wavelength range of roughly 530 nm to 580 nm. Further, each graph shows two peaks at roughly the same wavelength centers, 530 nm and 580 nm, and a third less pronounced peak at roughly 630 nm. As shown in FIGS. 3A and 3B, each of the peaks correspond to visible light in the red 301(1), (2), green 302(1), (2), and blue 302(1), (2) light spectrum.

FIGS. 3A and 3B each show Gaussian fitting displayed with 0 and 48 h UV light degraded on 30 μm polyethylene film. FIGS. 3A and 3B each demonstrate the changing fluorescence spectra and its corresponding Gaussian fits for visible light in the red 301(1), (2), green 302(1), (2), and blue 302(1), (2) light spectrum. For example, each graph shows three fluorescence populations (fit with Gaussian distributions) of stained fluorescence polyethylene being a peak red light 301(1), green light 302(1), and blue light 303(1) and the shift in peak red light 301(2), green light 302(2), and blue light 303(2) resulting from 48 hours of staining plastic material with Nile red. There is a clear shift in each of the three fluorescence spectra Gaussian populations in FIG. 3A to the three fluorescence spectra of Gaussian populations in FIG. 3B. The spectra center of mass for the visible color, red, shifts most significantly from FIG. 3A to FIG. 3B at increased film age of 48 hours. Nile red fluorescence, upon exposure to unaged polyethylene and polypropylene, have a consistent emission spectrum characterized by a three gaussian distribution. Conversely, when the fluorophore is exposed to more hydrophilic polymers, such as polyamide (PA) and polyethersulfone (PES), result in a single gaussian population.

FIG. 3C is a graph of example overlaid fluorescence spectra of different aged material (unaged, 2 h, 4 h, 6 h, 8 h, 12 h, 24 h, and 48 h aged: purple to green to red representing aging, at 30 μm thickness) stained with Nile red. In addition to the population changes in the spectra, a bathochromic shift in fluorescence signal is observed (FIG. 3C). The spectra substantially red shift, with an emission peak loss seen at 538 nm, when adsorbed onto the surface of aged polyethylene when compared with that of unaged polyethylene (FIG. 3C, unaged blue→green→aged red). The application of fluorophores as localized environmental sensors is well established in literature. That is, fluorophores, or fluorescent macromolecules, can actively change their photophysical properties depending on their environment. Due to the diverse nature of fluorophores and their photophysical properties, they can be used to sense a variety of conditions including solution pH, solution polarity, and biological toxins (i.e. toxic gases, heavy metals, etc.). Nile red is an example of an organic dye that is sensitive to its local environment's polarity with a well-documented shift in local polarity inducing a change in fluorescence emissions. Weathered PE experiences such a change in polarity as it ages. As PE photodegrades, the relatively hydrophobic surface of unaged polyethylene photo-oxidizes incorporates oxygen containing groups into the backbone, inducing polarity onto the polymer surface. Therefore, we propose the polarity changes of the polymer surface result in the observed shifts in the dye's fluorescence spectrum to lower energy (longer wavelength) emissions.

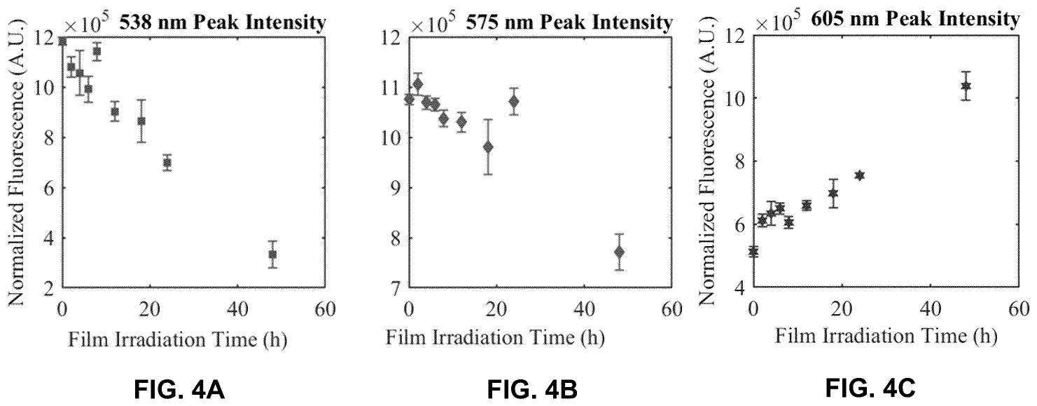

FIGS. 4A-4C each show peak intensities for 538 nm, 575 nm, and 605 nm, respectively, as a function of polyethylene film irradiation time (0-60). Tracking of each Gaussian fit peak across film ages. This figure shows the trends in peak intensity as film age increases. Peak intensity of 605 nm increases significantly as film irradiation time increases, while 538 nm and 575 nm peak intensities have a significant downward trend, except for 575 nm peak intensity at toughly 25 hours of film irradiation time. All fluorescence populations in FIGS. 4A-4C show consistent trends with film aging. The ratio of the 538 nm peak to the 605 nm peak can express both changes in peaks well.

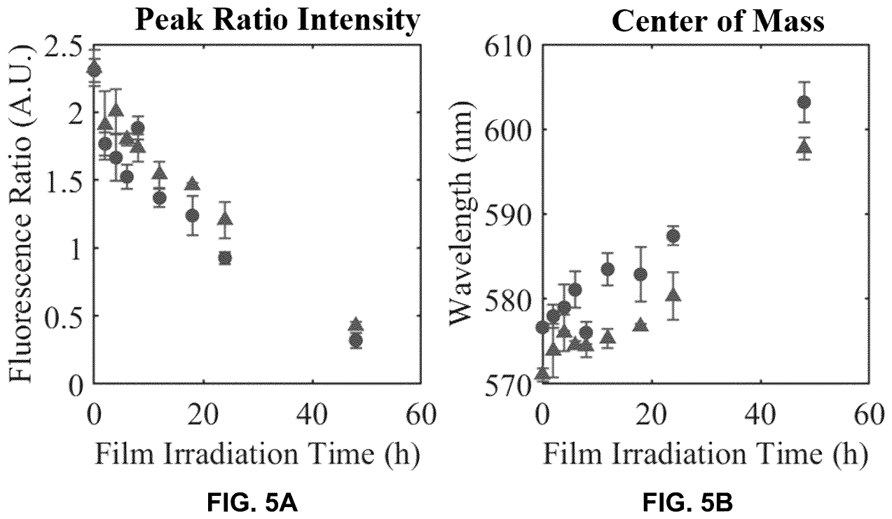

FIGS. 5A and 5B depict plots showing peak ratio intensity and center of mass changes as a function of film irradiation time. Blue circles in each figure represent 30 μm polyethylene and green triangles represent 50 μm polyethylene. Each of the 30 and 50 m PE films show congruent trends across film irradiation time. And peak ratio intensity and center of mass both trend linearly with polyethylene film age. This tracking method highlights Nile red's ability to interrogate the extent of oxidation in its local environment through the emission spectra shifts. Additionally, the trend seen in this peak ratio value across polyethylene irradiation time was consistent for both 30 μm (blue circle) and 50 μm (green triangle) PE and was found to be linear with markedly similar slope value, demonstrating the role of Nile red as a surface level age reporter. As seen in FIG. 5B, the change in the center of mass for fluorescence spectra across PE irradiation time for both 30 μm and 50 μm PE was also observed to be linear (Table 2 for fitting parameters). Both analysis methods provide a way to track Nile red's shift in fluorescence to lower energy populations due to the increase in surface polarity throughout PE degradation.

Another observation from FIGS. 5A and 5B is that both 30 μm (blue circle) and 50 μm (green triangle) polyethylene have statistically similar trends in fluorescence peak ratio (p=0.928) and center of mass (p=0.642) across plastic irradiation time, showing the results and photoaging and staining to be primarily a surface measurement that is independent of polymer thickness. This conclusion illustrates that the primary processes of polymer aging (i.e. photo and thermal oxidation, mechanical weathering, etc.) are initially limited to only the surface of degraded polymers. At later stages in aging, these processes can be seen to stretch further into the core of plastic films and solids, but is surface limited at relatively early stages. This highlights the value of an investigative technique, such as the one described herein, to monitor surface degradation. Other techniques for monitoring plastics do not achieve this feat because they do not succeed at monitoring either initial degradation phenomenon or surface dynamics.

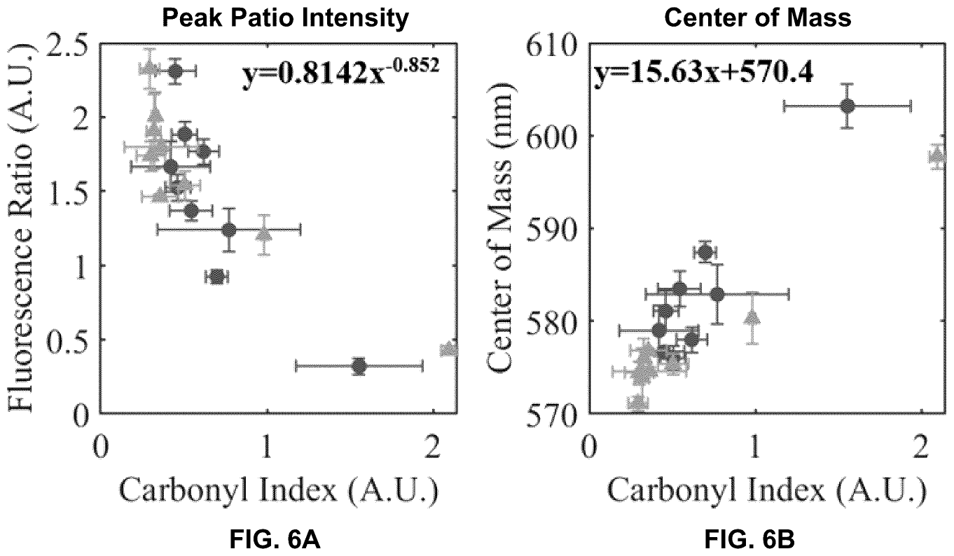

FIGS. 6A and 6B show 30 μm (blue circles) and 50 μm (green triangles) polyethylene film at peak ratio intensity and center of mass, respectively. Peak ratio intensity and center of mass both trend linearly with film age. As shown in FIG. 6B, center of mass for various wavelengths trends nearly linearly with carbonyl index. Greater changes are seen in peak ratio intensity at early stages of film irradiation.

FIGS. 6A and 6B illustrate the fluorescence peak ratio intensity and center of mass compared to carbonyl index values obtained from FTIR spectroscopy. The center of mass trends linearly with the carbonyl index for both 30 and 50 μm PE (FIG. 6B, linear fit equation: y=15.63x+570.4; Table 3). Interestingly, when looking at combined 30 μm and 50 μm PE, peak ratio intensity has relationship similar to an exponential decay with carbonyl index (y=0.8142x^{-0.852}, R²=0.6811), where initial time points of irradiation cause minimal changes in the carbonyl index (FIG. 6A) but large changes in fluorescence peak ratio. In previous literature, there is very little change observed in carbonyl index during the initial stages of aging, which suggests that this technique is not ideal for monitoring early degradation. Therefore, peak ratio analysis may serve as an important tool for differentiating small amounts of degradation that can overall have important ramifications to the processing and recyclability of these materials. Center of mass analysis, on the other hand, tracks more linearly (R² of 0.7935, Table 3 below) throughout the duration of aging with carbonyl index and also may be more applicable to other polymer types since not all polymers will have a multi-peak fluorescence spectra. More hydrophilic polymers, when stained with Nile red, have only a single gaussian fluorescence spectrum, thus making peak ratio intensity tracking unviable.

Beyond carbonyl index comparison, fluorescence intensities were also compared with bulk crystallinity and contact angle as illustrated below. Peak fluorescence intensity ratios and center of mass tracking are mostly linear with bulk crystallinity (e.g., Tables S4 and S5). An increase in crystallinity with irradiation is expected as photo-induced scission reactions in the amorphous regions cause repacking of the macromolecules into more crystalline microstructures. Therefore, crystallinity can be a substantial contributor or marker of the changes in chemical and physical properties of polyethylene that are observed in Nile red monitoring as it ages. Although DSC measures bulk crystallinity and not just surface crystallinity, photo-oxidation and repacking of polymer chain structures occurs initially at the surface level and so the early observed changes in crystallinity from DSC are likely predominantly at the surface level.

It should be noted that there were no noticeable changes in the onset of melting or melting temperature with plastic aging (Tables 6 and 7). Contact angle measurements collected for these samples are generally inconsistent and have large error within the measurements. This may be due to complications from changing physical properties of the polymer surface in addition to the chemical changes that govern water interactions with the plastic. Changes in crystallinity and surface roughness of these plastic films also change the wettability of the surfaces. This interplay between chemical and physical changes may be causing inconsistency in measurements. Overall, there is not an observable trend when comparing peak intensity ratios and center of mass with contact angle measurements. This suggests that contact angle goniometry may not be a strong tool for monitoring overall degradation at early stages in polyethylene films in the same way as the technique described herein, and further supports the value of this fluorescence technique as a more consistent approach to characterizing polymer degradation.

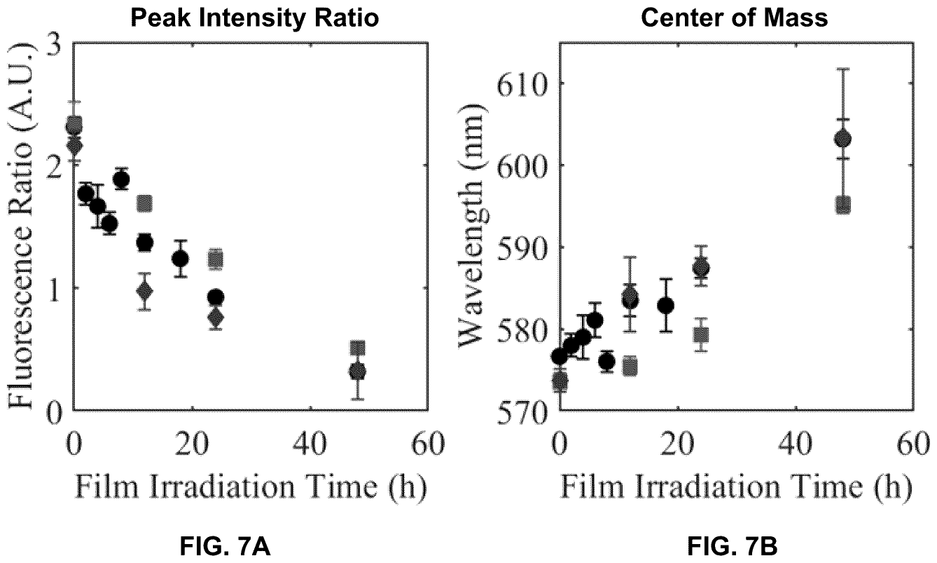

FIGS. 7A and 7B depict two example graphs: a first graph plotting a peak intensity ratio and a second graph mapping a center of mass of various wavelengths, in accordance with techniques of this disclosure. The performance of this method was assessed at different excitation wavelengths and storage times to evaluate the flexibility of this tool for other experimental constraints (e.g., other instrumentation). First, the storability of the staining was evaluated, with samples stained and measured freshly upon stain or upon being stored 11 months at room temperature in a dark, dry location. FIGS. 7A and 7B shows that the trends observed in stored and newly stained samples are statistically similar for both peak ratios and center of mass with irradiation time (p=0.905 and 0.434, respectively, with p-values representing probability of slope overlap due to variance). This supports the conclusion that Nile red staining is shelf stable and does not need to be conducted immediately after staining, especially when analyzed using the peak ratio analysis. Secondly, fluorescence measurements were explored where the excitation wavelength was shifted to 488 nm. Analysis of samples excited at different wavelengths (470 nm vs 488 nm) reveals statistically similar trends of the peak ratios and center of mass with degradation time (p=0.896 and 0.527, respectively). Testing the fluorescence response from an excitation at 488 nm was conducted in order to determine how response changes when exciting closer to the fluorescence peak. This can help guide better optimizing where to excite for unaged and aged samples in the future.

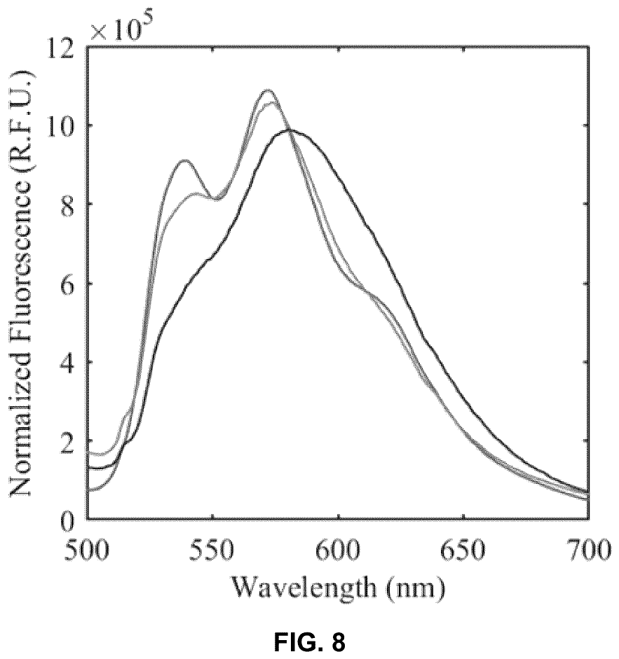

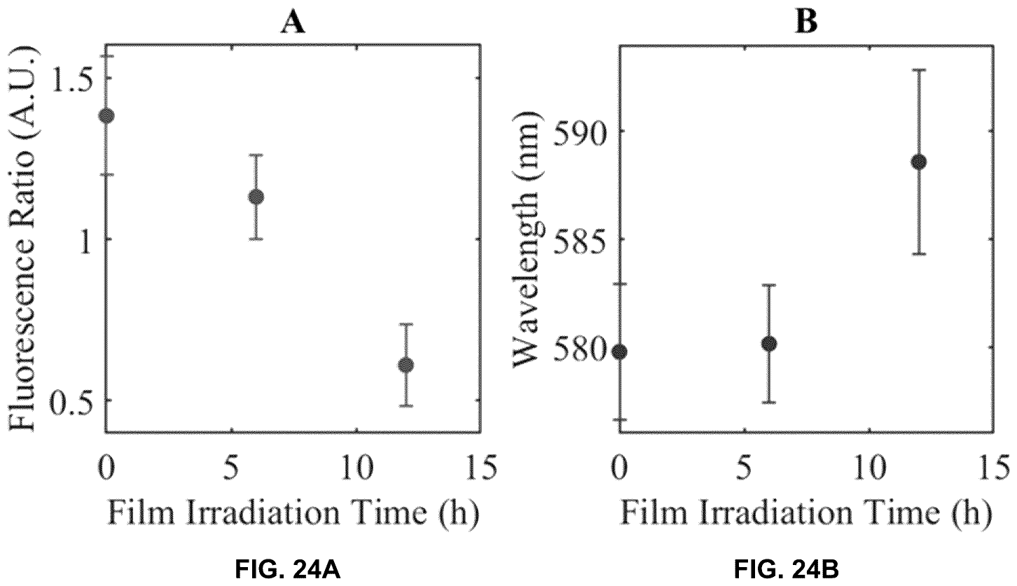

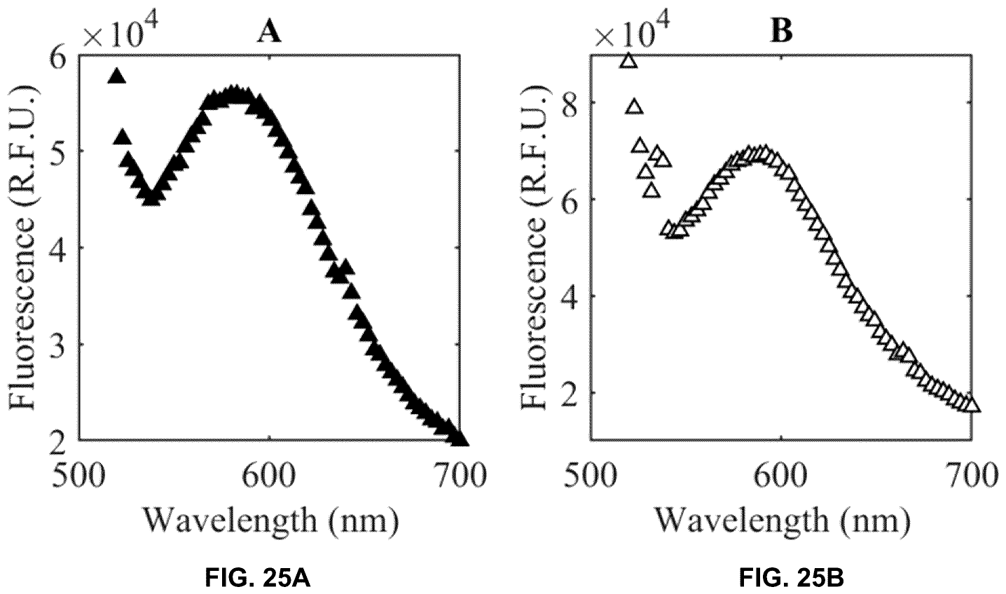

FIG. 8 is a graph of example fluorescence spectra at different ages using Nile red. This staining tool can also be applied to study the degradation of other polymer types, particularly hydrocarbon polymers. PP displays a similar three gaussian population to that of PE that shifts with photochemical aging (FIG. 8) and similar analyses can be conducted on it to track fluorescence changes as the polymer degrades. FIGS. 24A-B show that similar to PE, peak ratio fluorescence and center of mass tracking follow a similar trend, however there are also some differences between PE and PP in the correlation between fluorescence signatures and irradiation time. This variation is likely the result of varying degrees of degradation that are observed in these polymers under similar photon fluxes but indicate a similar calibration curve could be established for PP as had been demonstrated for PE. Beyond PP, the applicability of the staining tool was tested toward a substantially more hydrophilic polymer, poly-L-lactic acid (PLLA). Initial staining revealed substantially lower fluorescence signals, even when modifying stain solvent to water, because the increased hydrophilicity does not promote adsorption. Initial scans of stained PLLA show a weak fluorescence signal that appears to be mostly covered by the low levels of reflectance seen off the surface of the plastic samples (FIGS. 25A-B). The fluorescence yield of Nile red dye drops significantly with an increase in the hydrophilicity of its environment, including for other polymers like polyethylene terephthalate. The application of this staining technique, thus, depends on the ability to reduce the interference of reflectance for solid state analysis of PLLA. Nile red derivatives may be used for a wider variety of applications to different polarity environments.

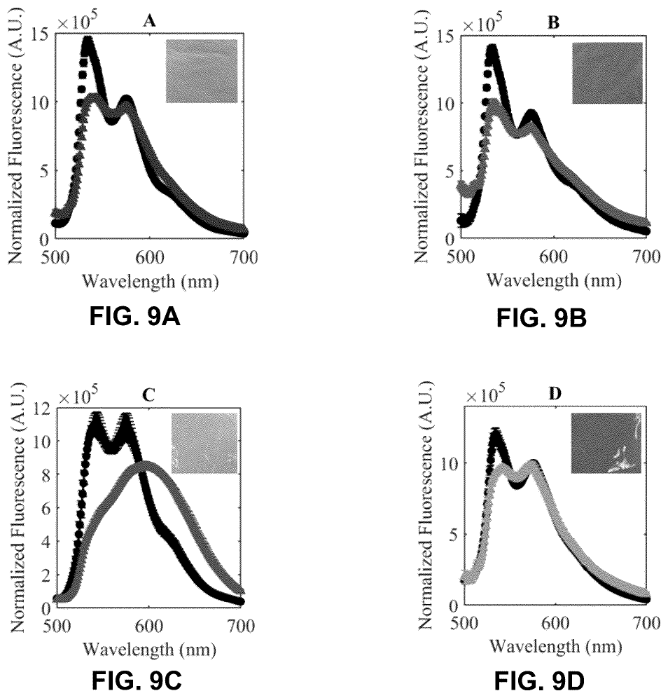

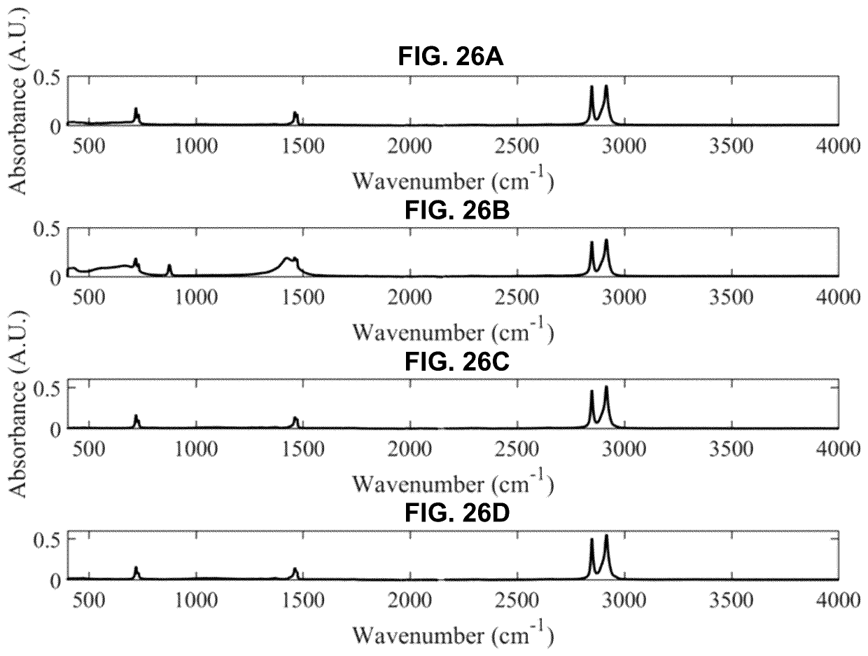

FIGS. 9A-9D are graphs of fluorescence spectra of different aged plastic samples. The effectiveness of this labeling and analysis strategy was evaluated for assessing commercial plastic samples with a variety of additives and fillers by staining four single-use, commercially available bags without and with photodegradation. The single-use bags included a brown/transparent grocery store bag (Cub Foods; Stillwater, MN), a gray/opaque shopping bag (Target; Minneapolis, MN), a colorless/transparent packaging bag (Solo Cup Company; Lake Forest, IL), and an opaque green food bag (Aldi's Little Salad Bar; Batavia, IL). FTIR spectroscopy was conducted on each of these samples and the plastics were all identified to be PE (FIGS. 26A-D).

The additives presented a challenge to differentiate between HDPE and LDPE from their FTIR spectra with methods that have been previously described but these bags are typically manufactured with LDPE. The fluorescence, as seen in FIGS. 9A-D, supports the conclusions that this dye staining technique can be used to collect information of commercially available samples that contain coloring dyes and plastic additives. The presence of dyes, additives, and/or varied manufacturing processes effect the observed gaussian populations seen in fluorescence spectra of the Cub Foods, Target bags, and Aldi's Little Salad Bar Bag (FIGS. 9A, 9B, and 9C colored spectra). For these samples, there is a distinct increase in the first gaussian population (539 nm) in the spectra when compared with that of the clear Solo Cups bag and the other clear PE samples used for this paper. This may be due to the increased presence of inorganic fillers in such as calcium carbonate in commercial plastics. The spectra of 48 h photochemically aged samples for the Cub Foods, Target, and Aldi's Little Salad Bar bags show differences from additive-free PE film samples used throughout the study, where the aged bag samples' spectra show similar gaussian populations to that of unaged additive-free films but have a lower fluorescence yield (FIGS. 9A, 9B, and 9C, colored spectra). The photoaged colorless commercial bag has fluorescence spectra similar to that of the additive-free PE used throughout this study.

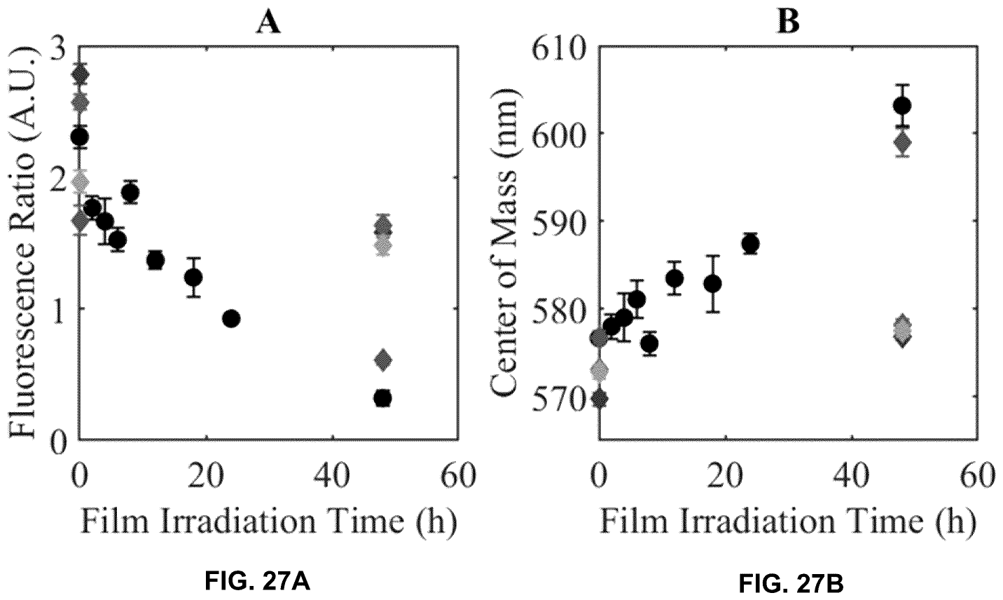

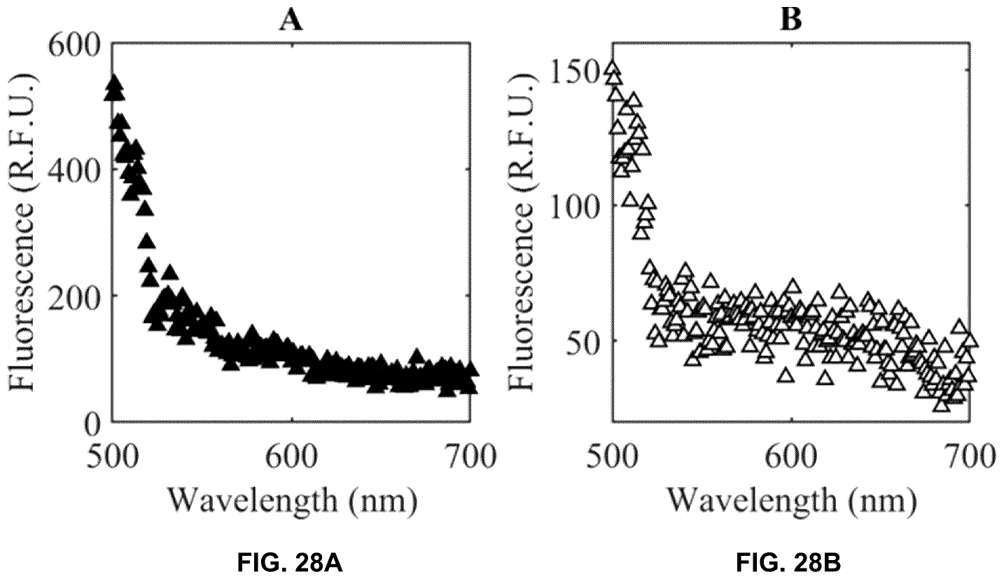

FIGS. 27A and 27B show how the fluorescence peak ratio and center of mass trends across irradiation time of these commercial samples compares with that of 30 μm additive-free PE. The commercial samples with additives show differences in changes of peak ratio and center of mass than that of the commercial samples without additives. Although variations to a calibration model for additive containing samples may improve detection, this data shows that the application of this technique and analyses was successful in a wider variety of plastic materials, including those with additives. Attempts were made to calculate a carbonyl index for these plastic samples but was difficult for the Cub Foods and Solo Cup Company bags and altogether not possible in the Target bags due to an overlap of an additive's signal with that of the PE reference peak that can be used to calculate a carbonyl index. Attempts were also made to stain black PE garbage bags, but these attempts yielded little result as no fluorescence signal was obtained (FIGS. 28A-B). This may be due to the presence of carbon black in these samples that may limit Nile red staining. It should be noted that background fluorescence signals from degraded plastics are not expected to interfere with this technique because the fluorescence occurs in plastics containing terephthalate (e.g., polyethylene terephthalate), and the fluorescence resulting from hydroxy-terephthalate (ex=325 nm; em=450 nm) does not overlap with the Nile red signals. Overall, this technique shows merit and its ability to analyze a variety of plastics with additives, including those that carbonyl index does not successfully interrogate. Further work can be explored in the future to help establish trends seen in these plastic samples.

The FIGS. 2-9B show that the results using Nile red appears to be independent of polyethylene film thickness. Further, peak ratio intensity monitoring can be useful for monitoring early stages of plastic film degradation (superior to center of mass and carbonyl index). Other data (not shown) supports longevity of techniques of the present disclosure with long-term storage of samples.

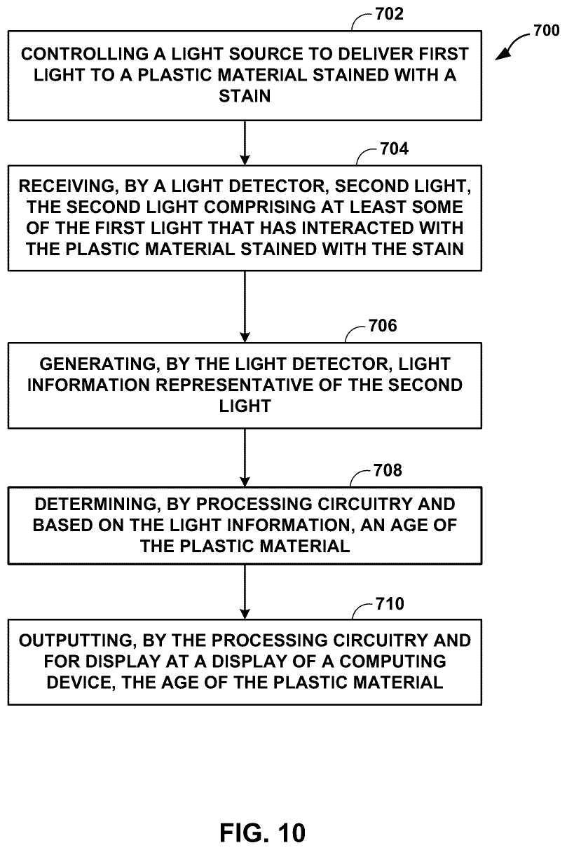

FIG. 10 is a flowchart illustrating a diagram for applying technical aspects of the present disclosure. The techniques of FIG. 10 are described with respect to system 10, but system 120, system 150, or other system or combination of devices may perform similar functions in other examples. The flowchart of the example of FIG. 10 begins with controlling a light source e.g., laser light 100) to deliver first light (e.g., first light 102) to a plastic material (e.g., plastic material 104, which may be any type of polymer, including polypropylene, polyethylene terephthalate, poly-lactic acid, and so on) stained with a stain (e.g., Nile red, such as a commercially available, lipophilic reporter fluorophore, and so on) (702). A light detector (e.g., a lens of camera 108) receives a second light (e.g., second light 106), the second light comprising at least some of the first light that has interacted with the plastic material stained with the stain (704). For example, after the first light interacts with the plastic material, fluorescent radiation is emitted from the plastic material-Nile red mixture. A filter (e.g., GFP dichroic) removes some of the fluorescent radiation, as discussed above, resulting in the second light.

The light detector (e.g., processing circuitry of camera 108) generates light information representative of the second light (706). For example, the light information representative of second light 106 may be substantially similar to as described with reference to FIGS. 2-6B. For example, generating light information may include converting RGB colors, received from the lens of camera 108, to corresponding respective wavelengths of the fluorescent radiation. Processing circuitry may determine an age of the plastic material based on the light information (708). For example, the ratio of wavelengths corresponding to green visible light may be compared against the wavelengths of corresponding red visible light. Further, the resultant ratio may match an experimental ratio stored within a database of the camera 108 or communicatively coupled to the camera 108. For example, the determined age may be based upon a match with the processed ratio and experimental ratio stored in the database. Processing circuitry may output the age of the plastic material and the processed data for display at a display (e.g., display 107 of camera 108) of a computing device (710).

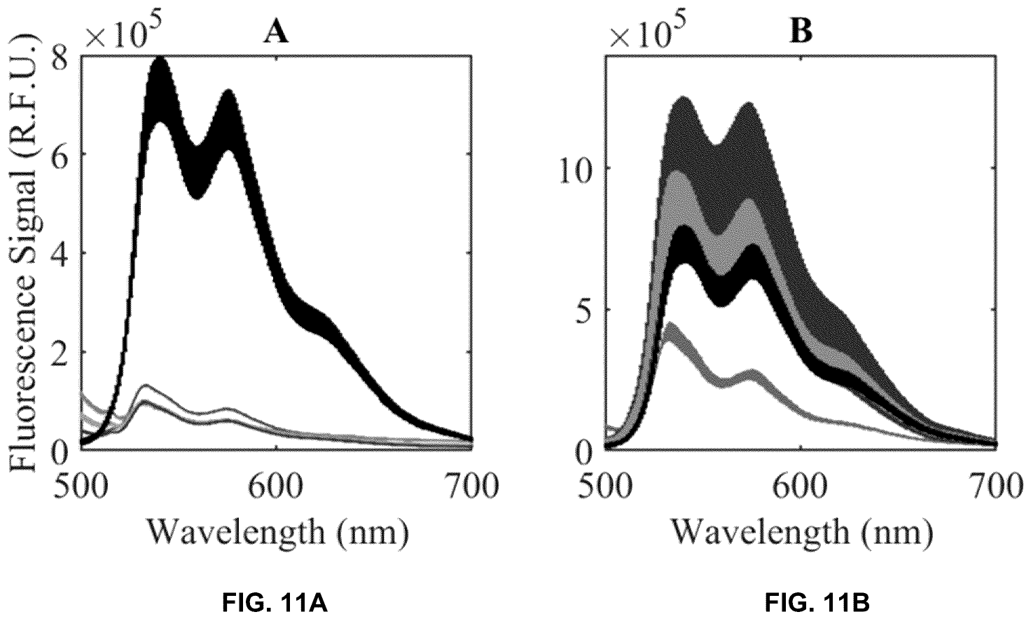

The following FIGS. 11A-31 provide explanation and additional data for the concepts described herein, including the discussed above. In some examples, stain time optimization can be determined for different materials or stains used to determine degradation of a material such as a polymer or combination of polymers.

FIG. 11A, 11B are graphs of example fluorescence spectra of for materials stained with Nile red for different durations. To determine the optimal time, or a desired time, for staining polymers, non-irradiated PE 30 μm films were placed in Nile red solutions ethanol for 1, 2, 3, 4, 8, 24, 48, and 72 hours. Fluorescent measurements were taken on triplicate samples and shown in FIGS. 11A and 11B. Shorter stain times (Figure FIG. 11A) yielded weak fluorescent signals, although they had relatively lower amounts of error and variability within the measurement. As stain time got longer (FIG. 11B), error increased substantially, which is likely the result of aggregation of the dye. Therefore, 8 hour (black spectra) was chosen as it has high signals with relative lower error. The spectra in FIG. 11A presented are 1 h (blue), 2 h (green), 3 h (yellow), 4 h (red), and 8 h (black) stain durations. The spectra in FIG. 11B presented for 24 h (blue), 48 h (green), 72 h (red), and 8 h (black) stain durations. Data points are plotted as the average of triplicate measurements with error bars reporting the standard deviation.

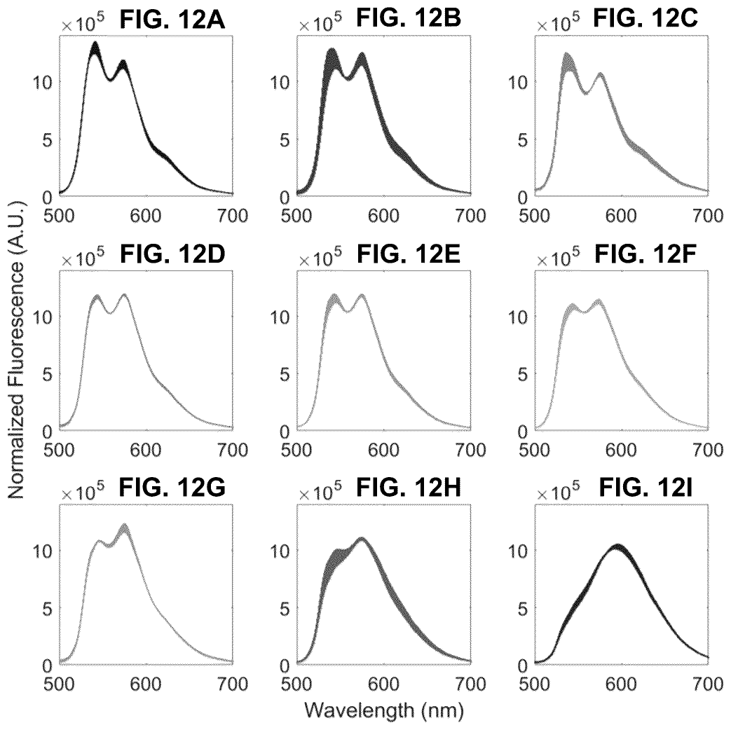

FIGS. 12A-12I are graphs of example fluorescence spectra of photo-degraded 30 um PE thin files stained with Nile red for different durations. Spectra presented are 0 h (A), 2 h (B), 4 h (C), 6 h (D), 8 h (E), 12 h (F), 18 h (G), 24 h (H), and 48 h (I) aged PE thin films. Data points are plotted as the average of normalized triplicate measurements with error bars reporting the standard deviation.

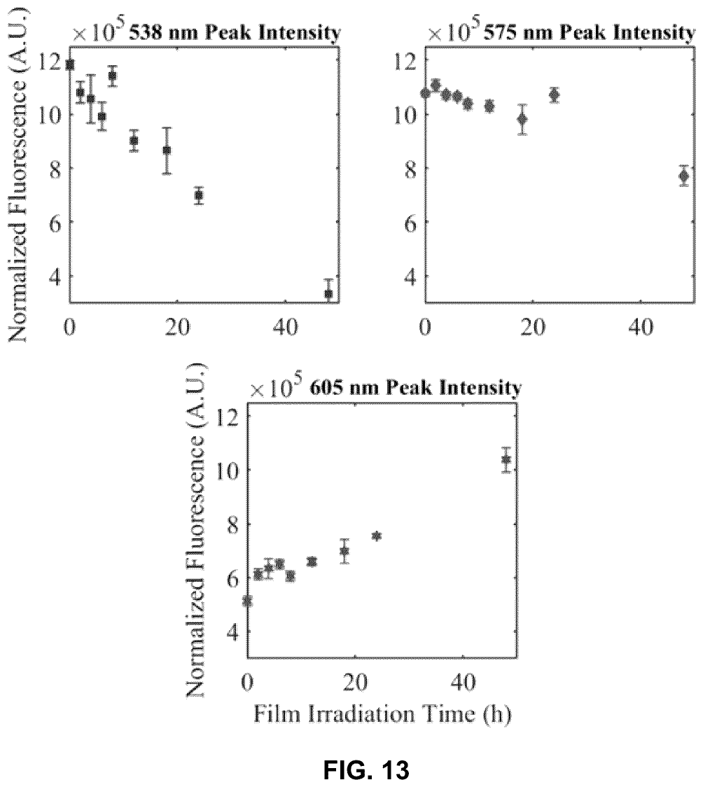

FIG. 13 includes graphs of fluorescence peak intensities at different wavelengths over time for polyethylene samples. The different wavelengths are 538 nm, 575 nm, and 605 nm, and each graph shows the fluorescence peak intensity changes with irradiation time of 30 μm PE samples. Peak locations in each graph correspond to the locations of the gaussian distributions as seen in FIG. 3A. All data points represent the average of normalized triplicate measurements with error bars representing the standard deviation.

TABLE 1
Peak Ratio with Film Irradiation Time Linear
Fitting Parameters with 470 nm Excitation

| PE Film Thickness | m (h⁻¹) | b | R² |
|---|---|---|---|
| 30 μm | −0.0361 | 1.934 | 0.8833 |
| 50 μm | −0.0356 | 2.084 | 0.9585 |

TABLE 2
Center of Mass with Film Irradiation Time Linear
Fitting Parameters with 470 nm Excitation.

| PE Film Thickness | m (nm/h) | b (nm) | R² |
|---|---|---|---|
| 30 μm | 0.5378 | 575.8 | 0.9356 |
| 50 μm | 0.5006 | 571.0 | 0.9164 |

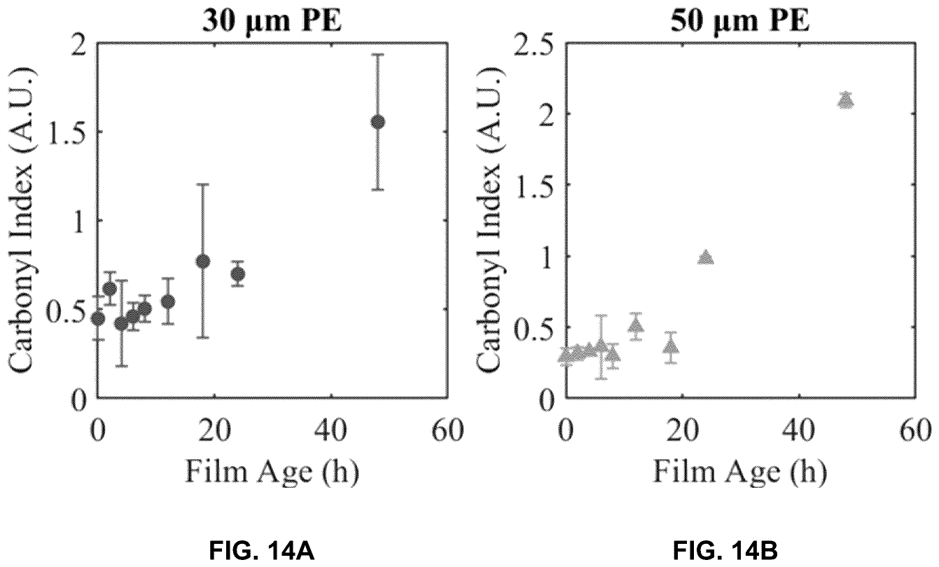

FIG. 14A, 14B are graphs of example carbonyl index value changes with irradiation time for different thickness samples. In the example of FIGS. 14A and 14B, carbonyl index value change with irradiation time of in both 30 and 50 μm PE thin films. Each data point represents the average of triplicate measurements with error bars representing the standard deviation.

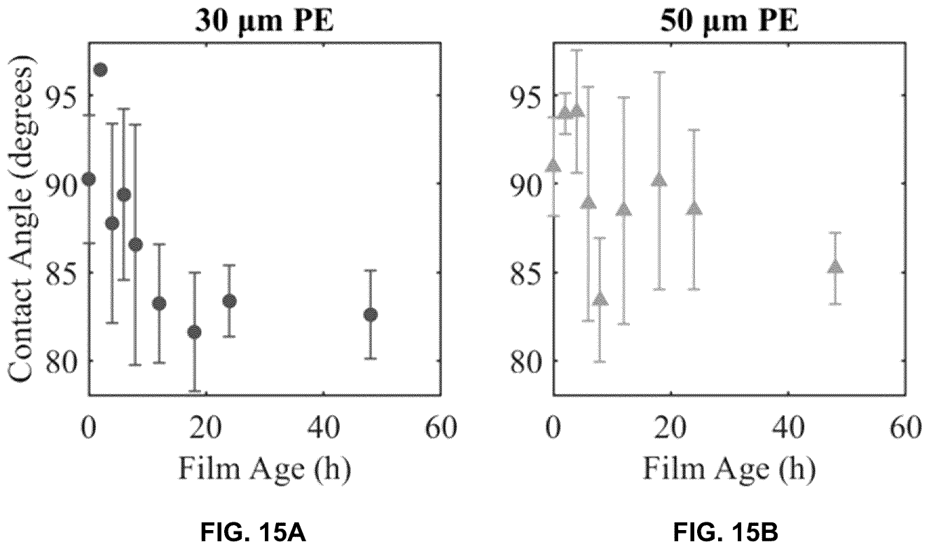

FIG. 15A, 15B are graphs of example contact angle measurement changes with irradiation time for different thickness samples. In the example of FIGS. 15A and 15B, the contact angle measurement changes with irradiation time for both 30 (blue circles) and 50 (orange triangles) pm PE thin films. Each data point represents the average of triplicate measurements with error bars representing the standard deviation.

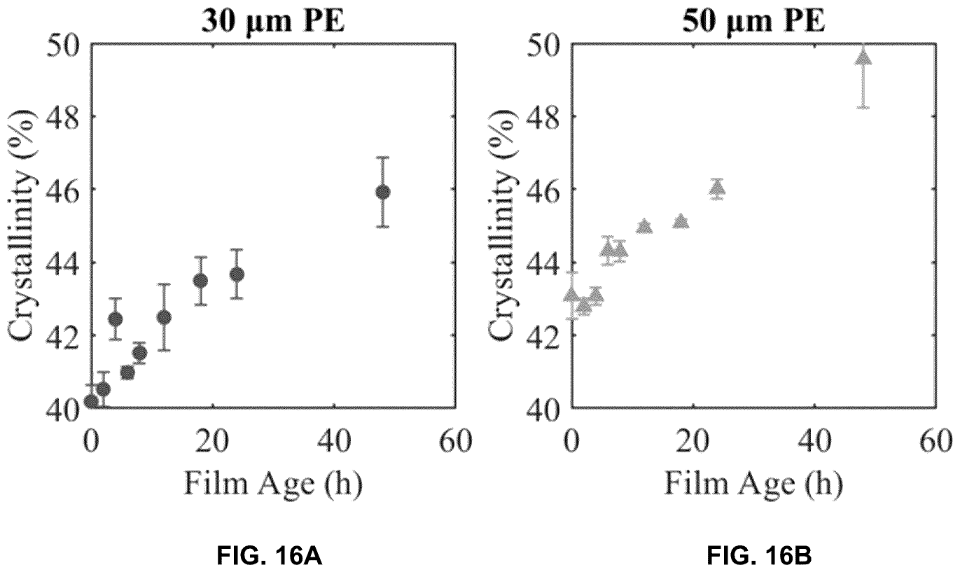

FIG. 16A, 16B are graphs of example bulk crystallinity changes with irradiation time for different thickness samples. In the example of FIGS. 16A and 16B, the bulk crystallinity changes with irradiation time for both 30 (blue circles) and 50 (orange triangles) pm PE thin films. Each data point represents the average of triplicate measurements with error bars representing the standard deviation.

TABLE 3
Center of Mass with Carbonyl Index Linear
Fitting Parameters with 470 nm Excitation.

| PE Film Thickness | m (nm/h) | b (nm) | R² |
|---|---|---|---|
| 30 μm | 22.28 | 568.8 | 0.8789 |
| 50 μm | 12.93 | 569.8 | 0.9545 |
| Combined | 15.63 | 570.4 | 0.7935 |

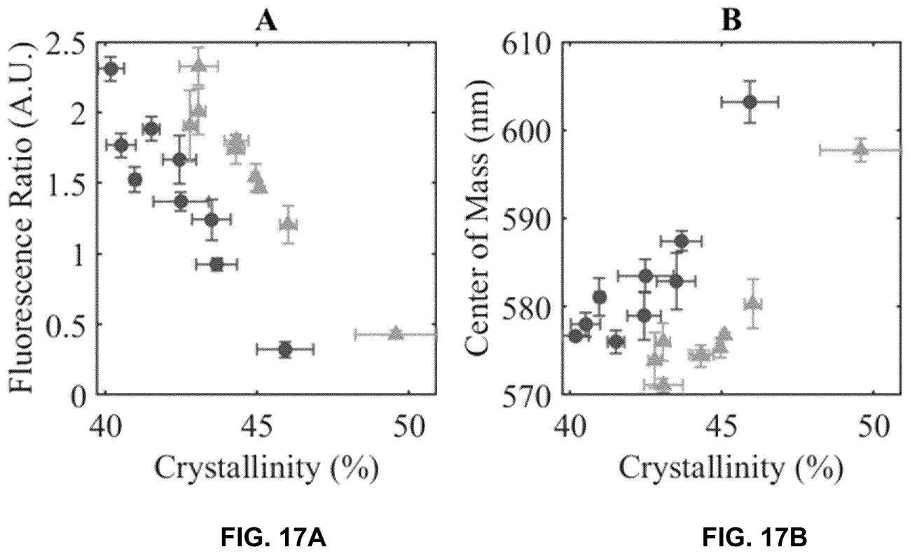

FIG. 17A, 17B are graphs of example peak fluorescence intensity ratios and center of masses compared with bulk crystallinity across film irradiation for different thickness samples. In the example of FIGS. 17A and 17B, 539 and 605 nm Peak Fluorescence Intensity Ratio (A) and Center of Mass (B) changes are compared with bulk crystallinity across film irradiation for both 30 μm (blue, circle) and 50 μm (orange, triangle). Data points show the fluorescence peak intensity ratio and corresponding bulk crystallinity measure of each aged polyethylene sample with 0 h aged PE located in the upper left region with signal moving to 48 h aged PE located in the lower right region in FIG. 17A. Each data point represents the average of triplicate measurements for each technique with error bars representing the standard deviation in both the x and y directions.

TABLE 4
Peak Ratio Intensity with % Crystallinity Linear
Fitting Parameters with 470 nm Excitation.

| PE Film Thickness | m (nm/h) | b (nm) | R² |
|---|---|---|---|
| 30 μm | −0.2973 | 14.03 | 0.8724 |
| 50 μm | −0.2545 | 13.00 | 0.9389 |
| Combined | −0.1705 | 8.952 | 0.4927 |

TABLE 5
Center of Mass with % Crystallinity Linear
Fitting Parameters with 470 nm Excitation.

| PE Film Thickness | m (nm/h) | b (nm) | R² |
|---|---|---|---|
| 30 μm | 4.117 | 408.7 | 0.7989 |
| 50 μm | 3.642 | 419.14 | 0.8789 |
| Combined | 1.980 | 494.2 | 0.2915 |

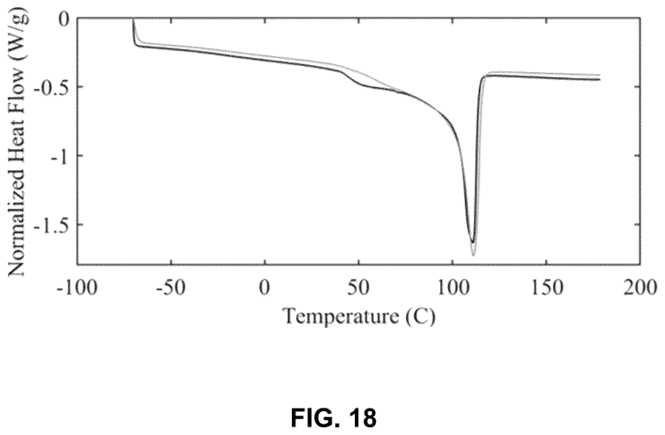

FIG. 18 is a graph of an example melting curve of 30 um polyethylene as measured by differential scanning calorimetry. Scans shown are 0 h, unaged polyethylene (black), and 48 h, aged polyethylene (orange).

TABLE 6
Melting Temperature and Onset of Melting of 30 μm Polyethylene
Samples. Error represents the standard deviation
from three replicate measurements.

| Irradiation Time (h) | Melting Temperature (° C.) | Onset of Melting(° C.) |
|---|---|---|
| 0 | 111.38 ± 0.51 | 101.68 ± 0.21 |
| 2 | 110.74 ± 0.57 | 102.18 ± 0.18 |
| 4 | 110.20 ± 0.25 | 102.08 ± 0.04 |
| 6 | 110.51 ± 0.23 | 101.96 ± 0.08 |
| 8 | 111.05 ± 0.67 | 101.75 ± 0.37 |
| 12 | 111.31 ± 0.08 | 101.49 ± 0.45 |
| 24 | 110.55 ± 0.24 | 102.17 ± 0.10 |
| 48 | 110.17 ± 0.28 | 102.41 ± 0.15 |

TABLE 7
Melting Temperature and Onset of Melting of 50 μm Polyethylene
Samples. Error represents the standard deviation
from three replicate measurements.

| Irradiation Time (h) | Melting Temperature (° C.) | Onset of Melting(° C.) |
|---|---|---|
| 0 | 111.27 ± 0.67 | 103.86 ± 0.78 |
| 2 | 112.47 ± 0.20 | 103.80 ± 0.16 |
| 4 | 111.61 ± 0.20 | 103.93 ± 0.25 |
| 6 | 111.75 ± 0.18 | 103.84 ± 0.02 |
| 8 | 111.80 ± 0.10 | 103.16 ± 0.18 |
| 12 | 112.02 ± 0.17 | 103.30 ± 0.12 |
| 24 | 112.55 ± 0.47 | 102.80 ± 1.00 |
| 48 | 112.71 ± 0.26 | 102.85 ± 0.89 |

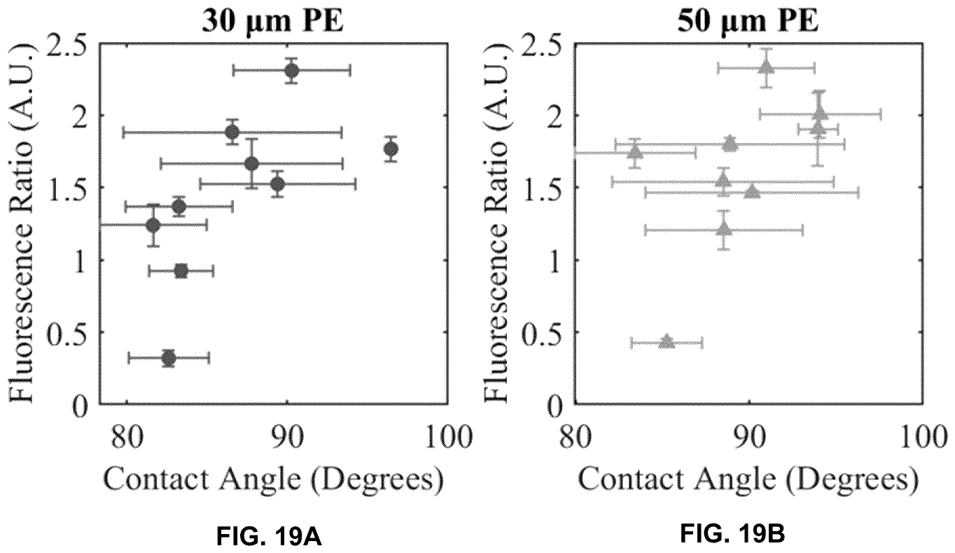

FIGS. 19A, 19B are graphs of example peak fluorescence intensity ratio changes compared with contact angle measurements across film irradiation of both aged 30 μm and 50 μm polyethylene films. Data points show the fluorescence peak intensity ratio and corresponding contact angle measurement of each aged polyethylene sample with 0 h aged PE located in the upper right region with signal moving to 48 h aged PE located in the lower left region for both figures. Each data point represents the average of triplicate measurements for each technique with error bars representing the standard deviation in both the x and y directions.

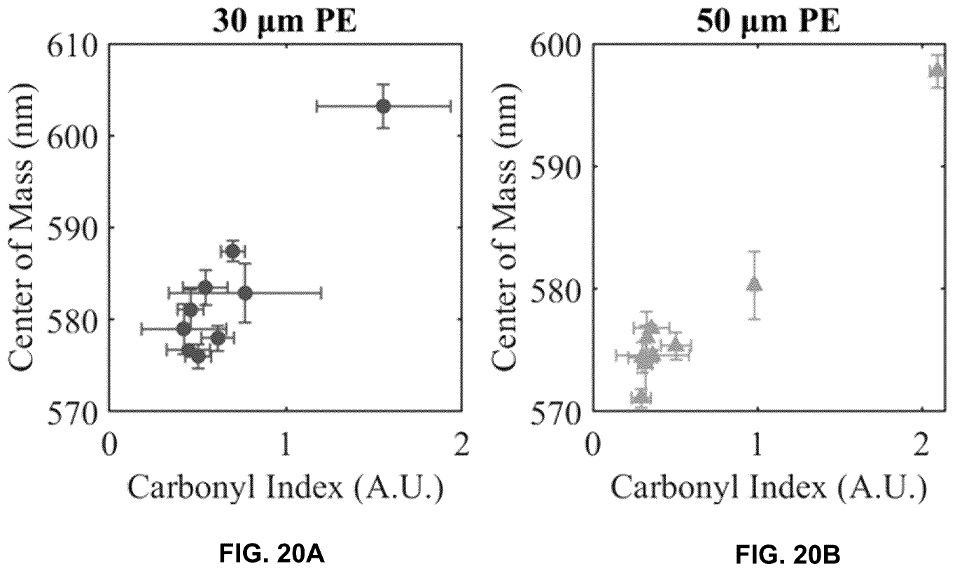

FIGS. 20A, 20B are graphs of example center of mass changes compared with contact angle measurements across film irradiation of both aged 30 μm and 50 μm polyethylene films. Data points show the center of mass and corresponding contact angle measurement of each aged polyethylene sample with 0 h aged PE located in the upper left region with signal moving to 48 h aged PE located in the lower right region of each figure. Each data point represents the average of triplicate measurements for each technique with error bars representing the standard deviation in both the x and y directions. In some examples, the center of mass (COM) may reflect the average perceived color of the image. This COM may be computed as:

$${x ( nm )}_{COM} = \frac{{\sum }_{i} {x ( nm )}_{i} \times flourescence {Intensity}_{i}}{{\sum }_{i} flourescence {Intensity}_{i}} \qquad ( 2 )$$

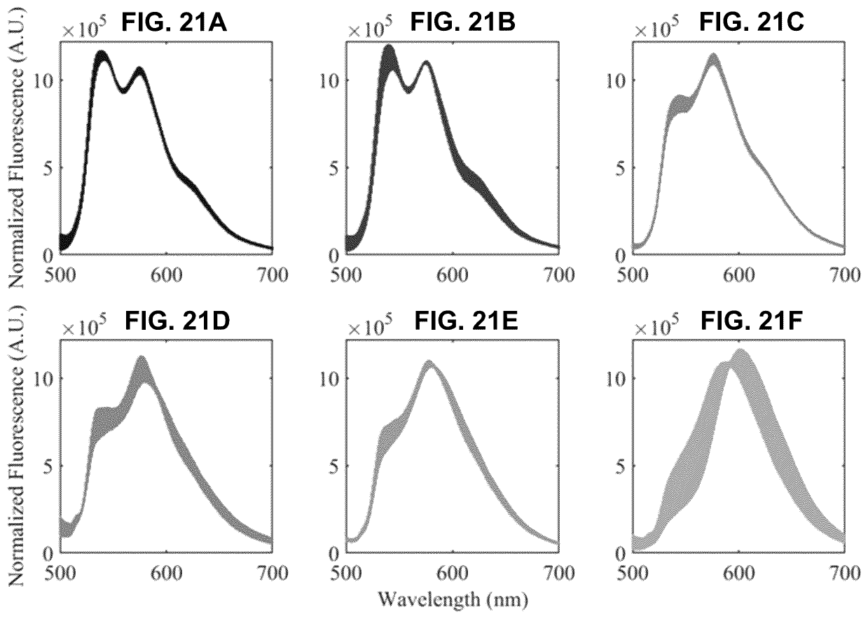

FIGS. 21A-21F are graphs of example fluorescence spectra of thermally degraded PE thin films stained with Nile red. Spectra presented are 24 h (A), 48 h (B), 72 h (C), 192 h (D), 264 h (E), and 336 h (F) aged films. Data points are plotted as the average of normalized triplicate measurements with error bars reporting the standard deviation.

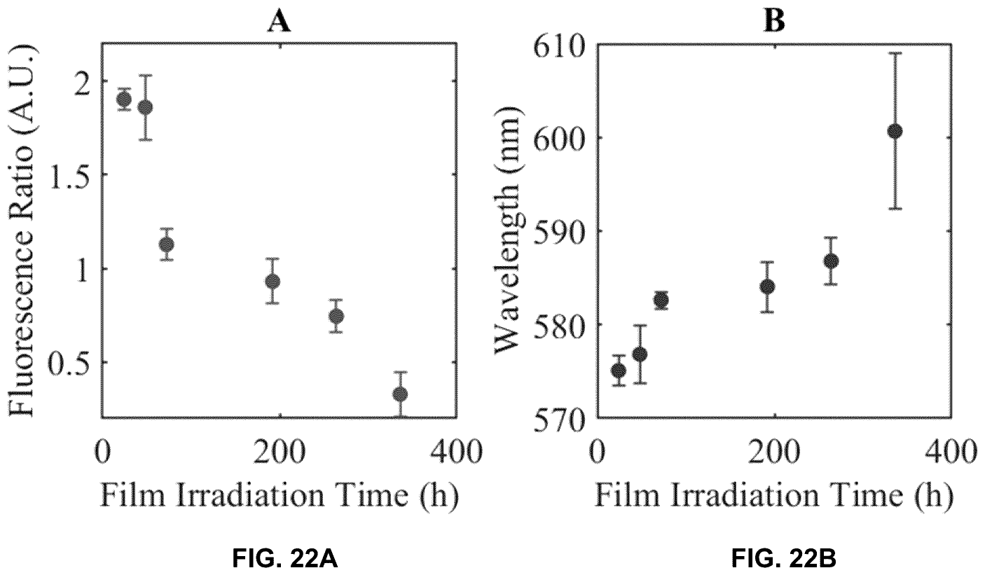

FIGS. 22A, 22B are graphs of example peak fluorescence intensity ratios (FIG. 22A) and center of mass (FIG. 22B) changes with film irradiation for thermally degraded PE samples. All data points are the average of triplicate measurements with error representing the standard deviation.

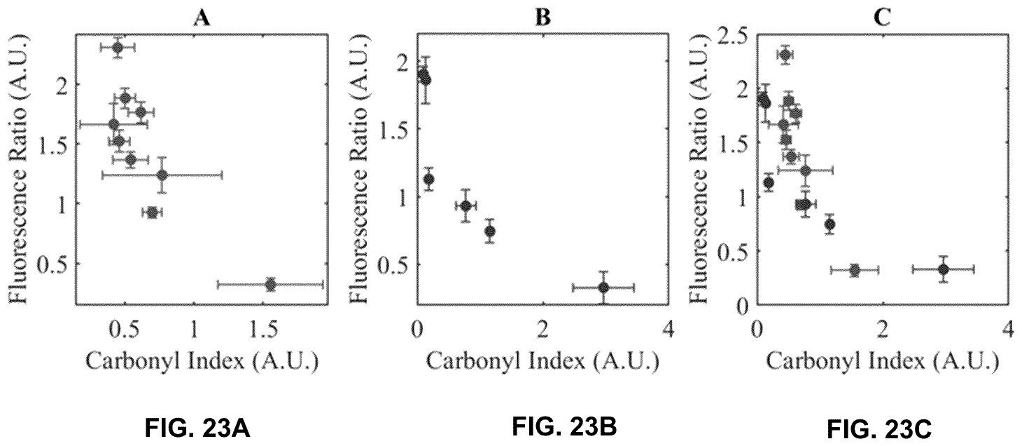

FIGS. 23A, 22B, and 23C are graphs of example peak fluorescence intensity ratio changes of photo-degraded (blue) (A) and thermally degraded (red) (B) compared with carbonyl index of PE with overlaid data for visual comparison of trends (C). All data points are the average of triplicate measurements with error representing the standard deviation. The same characteristic shift in fluorescence populations is observed with thermal degradation (FIGS. 21A-F and 22A-B) and peak ratio tracking shows similar correlation to that of photo-oxidative degradation as it relates to carbonyl index (FIG. 23A-C). These observed trends support the conclusion that this technique used to track degradation of PE in general, no matter the external stressor. This result is consistent with the literature findings that have found that thermal and photo-degradation both show the production of oxygen containing functional groups in degraded plastics. There can be a difference in oxidative products for thermal and photo-degradation, but overall these processes result in similar carbonyl index values.

FIGS. 24A, 24B are graphs of example 539 nm and 605 nm peak fluorescence intensity ratio (A) and center of mass (B) changes with film irradiation for photo-degraded PP thin film samples. All data points are the average of triplicate measurements with error representing the standard deviation.

FIGS. 25A, 25B are graphs of example fluorescence Spectra of stained poly-L-lactic acid films at different excitation wavelengths. Excitation wavelengths are 470 nm (A) and 488 nm (B).

FIGS. 26A-26D are graphs of example FTIR absorbance measurements of unaged commercial plastic samples. Plastic samples presented are different commercial plastic bags, including: Cub Foods bag (A), Target bag (B), Solo Cup Company bag (C), and Aldi's Little Salad Bag (D).

FIGS. 27A, 27B are graphs of example 539 nm and 605 nm peak fluorescence intensity ratio (A) and center of mass changes (B) with film irradiation time for different commercial plastic samples. The samples of FIGS. 27A, 27B include a 30 μm thick sample of PE (black, circle), Cub Foods bag (red, diamond), Target bag (green, diamond), Solo Cup Company (blue, diamond), and Aldi's Little Salad Bar Bag (orange diamond). All data points are the average of triplicate measurements with error representing the standard deviation.

FIGS. 28A, 28B are graphs of example raw fluorescence signals of stained and unirradiated sample at 0 h (A) and irradiated at 48 h (B) black plastic garbage bag samples. The signal obtained is attributed to reflectance obtained while doing solid state fluorescence.

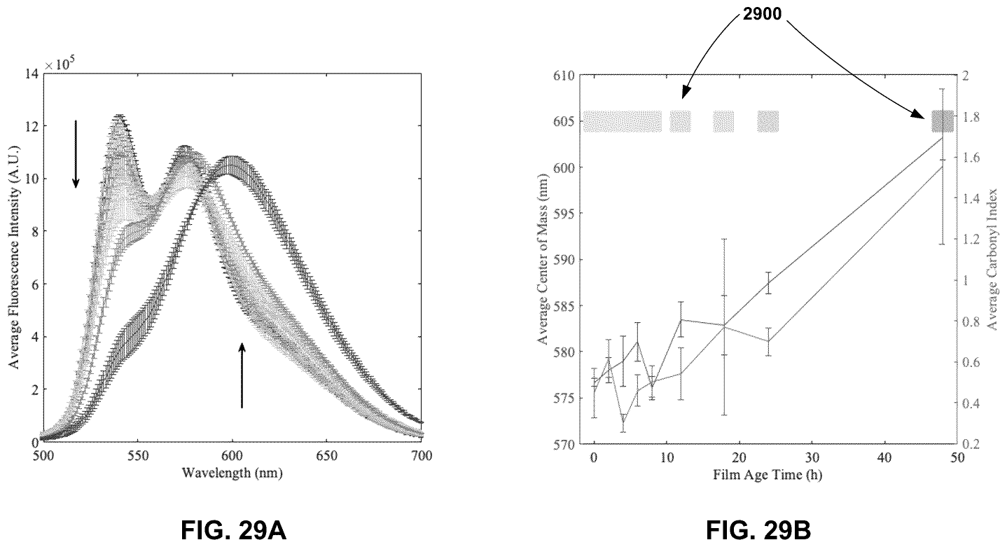

FIG. 29A is a graph of average fluorescence intensity for different wavelengths, and FIG. 29B is a graph of average center of mass and average carbonyl index for different film ages. As described herein, responder dyes and collected imaging data at certain wavelengths can be analyzed to determine aging information for different polymer samples. In other words, aging information can be derived from perceived colors reported during the imaging process.

In general, spectroscopic analysis demonstrates that the age to color relationship corresponds, or follows, the age to oxidation relationship for plastics subject to a responder dye (e.g., Nile red). Oxidation can be determined by carbonyl index (CI) measurements performed using Fourier-transform infrared spectroscopy (FTIR) using ASTM F2102-17. This oxidation thus measures the incorporation of oxygen into plastics as they age. The spectroscopic Center of Mass (COM) as described above in equation 2 can then reflect the average perceived color.

FIG. 29A shows the average fluorescence intensity of different wavelengths for different colors. FIG. 29B illustrates the average center of mass on the left vertical axis and the average carbonyl index on the right vertical axis, with the film age in hours on the x-axis. Generally, the center of mass and the average carbonyl index increases with age of the plastic. The perceived color 2900 reflects this change, with a lighter yellow color occurring near time zero, and the yellow color darkening towards orange at 50 hours.

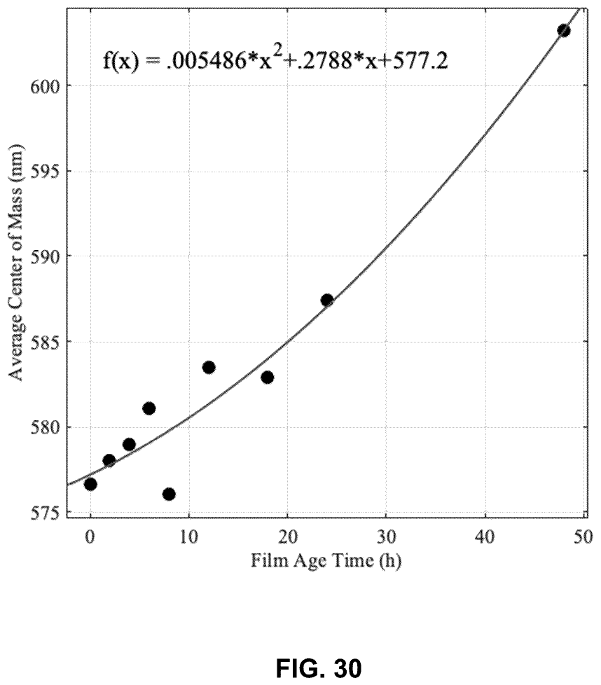

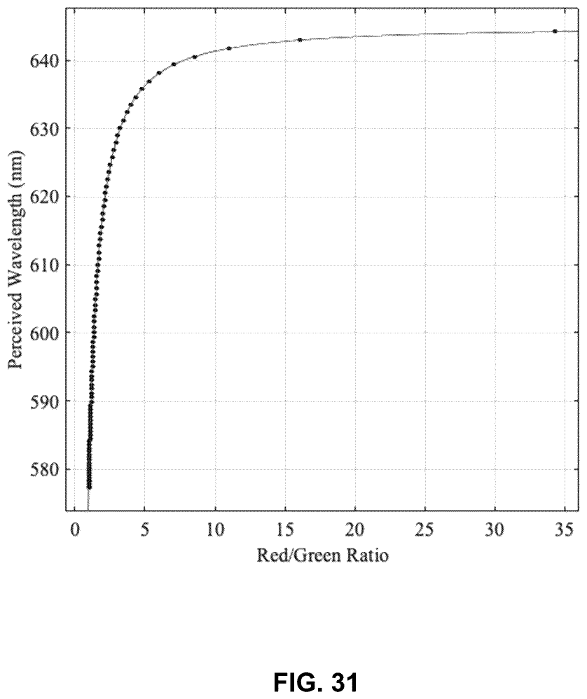

The detected light from the sample is at various wavelengths, which is converted to RGB data (which can be output as a picture). As discussed above, carbonyl index and fluorescence data can be fit to predict the effective perceived wavelength. However, it is not straightforward to convert RGB data back to the wavelength in order to extract information. Put another way, there is no direct analysis route to recover wavelength information from RGB data to then directly extrapolate to plastic age. However, in some examples, the system can fit the data to approximate wavelength and then determine film age as discussed herein and also in FIGS. 30 and 31.

FIG. 30 is a graph of average center of mass over different film ages. The system may fit certain data in order to recover wavelength information from RGB data. For example, the system can generate wavelength to RGB data from 577 nm to 630 nm. This range corresponds to the range for Nile red in this example. RGB corresponds to 630 nm, 532, and 456 nm, respectively. Above 630 nm, the signal is variably shaded red with no green. Above 630 nm, the maximum carbonyl index is also reached.

The system can then determine the ratio of green to red in the RGB code from the images. In some examples, Nile red may only require blue color reporting in bleed through or if light makes it through the dichroic filter, which means that ignoring blue data mechanically filters the data. As shown in the graph of FIG. 30, the system can generate a polynomial fit to ascertain approximate wavelength according to the average center of mass and film age time. The system can then use the wavelength fit to determine the effective age based on the carbonyl index for the sample.

FIG. 31 is a graph of perceived wavelength over different ratios of red to green from RGB data. As shown in the example of FIG. 31, the perceived wavelength, and thus perceived color, can be determined from the red to green ratio in the RGB data. In general, the perceived color reported in wavelength can be determined by calculating the COM from spectroscopic data collected on aged polyethylene (PE) or other plastics exposed to Nile red.

The system can generate a database of wavelengths characterizing all of the possible perceived colors that may occur on the surface of aged plastics. This databased of all possible wavelengths can be converted into their RGB code using wavelength2color.m (from Matlab), for example. The system can determine the ratio of red to green values from imaging data to generate a distinct set of values that describes the exact red to green relationship with respect to wavelength as shown in FIG. 31. This red to green ratio fit to relate RGB code to perceived wavelength can overcome a limitation of not being able to convert RGB data back to wavelength, and this identify specific colors that were detected. This can work in certain situations because blue may not be needed to describe the color of the sample. The system can also translate the perceived wavelength into a degree of oxidation, and this aging. This aging data can be generated by the system in various formats, such as an image with a color bar to indicate regions of variable aging within the sample or from different samples in the same imaging data.

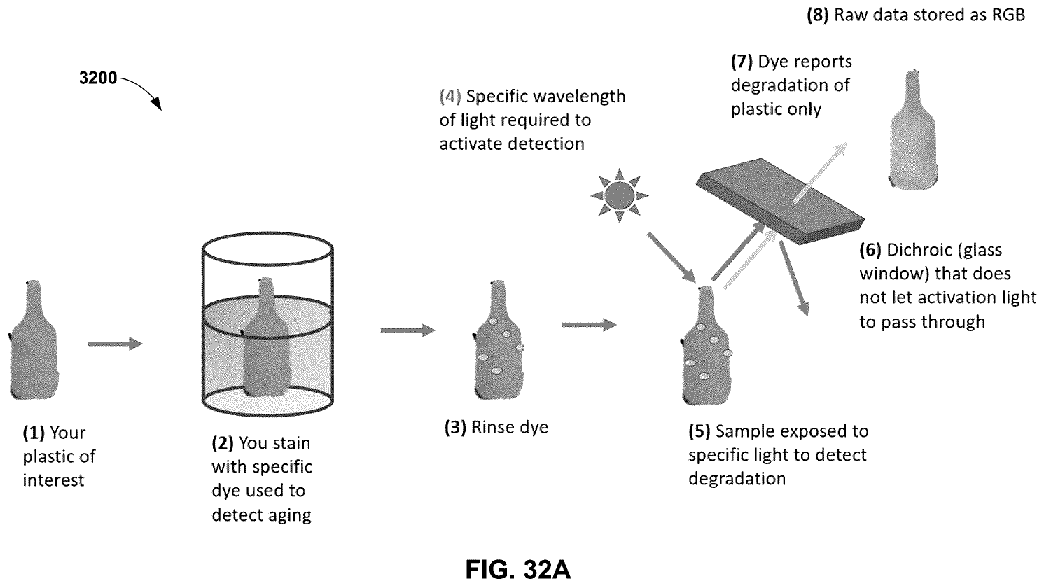

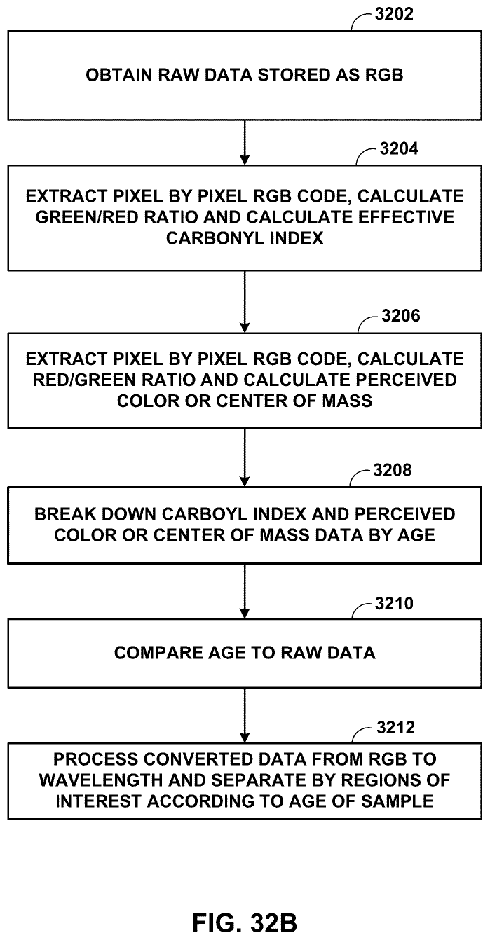

FIG. 32A is a conceptual diagram of an example technique for obtaining image data for a sample of plastic subject to a specific dye. As shown in the example technique 3200 of FIG. 32A, the technique may start with selecting the plastic sample of interest (1). Then, the user can stan the sample with the specific responder dye that can be indicative of aging of the sample (2). The dye is then rinsed off (3) before the system (e.g., system 10 or 120) directs a specific wavelength of light to activate detection (4). The sample is exposed to this specific wavelength of light to detect the degradation that occurred so far (5). Some of the light emitted by the sample goes toward a dichroic glass (e.g., a glass window that acts like a filter) and is passed through instead of the specific wavelength of light (6). The light received by a light sensor is indicative of the dye reports degradation of the plastic only (7), and the light sensor stores the image data as RGB data and/or transmits the RGB data to another device for analysis (8).

FIG. 32B is a flow chart of an example technique for determining age of samples from obtained image data. In the example of FIG. 32B, RGB data collected from image data in the technique of FIG. 32A or as otherwise described herein may be used. Processing circuitry 152 of system 150 will be described as an example, by other devices or systems may perform some or all of the aspects of the technique in FIG. 32B.

As shown in FIG. 32B, processing circuitry 152 may obtain the rate data stored as RGB data (3202). Processing circuitry 152 may then extract, pixel by pixel, RGB code, calculate the green to red ratio, and calculate the effective carbonyl index (3204). Processing circuitry 152 may use the green to red ratio to extract the specific wavelength from the image data and then calculate the effective carbonyl index (e.g., the extent oxidation) using the extracted wavelength. Processing circuitry 152 may generate images or graphs of the age data to identify the ages of one or more samples within the image data. Processing circuitry 152 may also extract, pixel by pixel, RGB code, calculate the red to green ratio, and calculate the perceived color or center of mass (3206). Processing circuitry 152 may use the red to green ratio to extract the specific wavelength from the perceived color or center of mass. Processing circuitry 152 may generate images or graphs of the age data to identify the ages of one or more samples within the image data. Although both of steps 3204 and 3206 are described to provide additional data regarding the sample, in other examples, only one of steps 3204 or 3206 may be performed. In other examples, steps 3204 and 3206 may be performed in different order, or simultaneously.

Processing circuitry 152 may determine the age of the plastic samples in the image data by at least one of breaking the carbonyl index down by age and/or using the extracted center of mass wavelength to break down data by age (e.g., the extent oxidation) (3208). Processing circuitry 152 may generate graphs, numerical data, identifying regions within an image of the samples, or any other information to the user based on the age determination in this technique.

In some examples, processing circuitry 152 may compare the determined age or other computed elements to the raw data for comparison (3210). Then, processing circuitry 152 may then process the converted data from RGB to wavelength and separate by regions of interested according to the age of the sample or samples within the obtained images (3212). For example, processing circuitry 152 may generate multiple different images that each indicate the areas of the original image that correspond to specific respective ages. Or, a single image that is color coded or otherwise indicates the ages of various plastics within the image may be generated. In any case, processing circuitry 152 may generate for display images or other information that a user, or system, can process to determine the age of the samples of plastic of interest. The system may sort, processor, or otherwise take additional action on the samples based on the determined ages.

As described herein, Nile red is used as one example responder dye. However, other responder dyes (Table 8) may be used in other examples that may correspond to respective wavelengths. Generally, a system may be configured to provide light at excitation wavelengths from 450 nm to 690 nm in some examples, and detect emission wavelengths from 480 nm to 800 nm. Various narrower ranges of wavelengths or specific wavelengths may be used within these ranges as appropriate for the responder dye used for any sample.

TABLE 8
Example responder dyes that may be applied to plastics
and used to determine age as described herein.

| | Excitation | Excitation | Emission |
|---|---|---|---|
| Dye Name | (nm) | region (nm) | (nm) |
| Nile red | 470 | 460-490 | 490-700 |
| Acridine Orange | 475 | 465-495 | 490-700 |
| Safranin O | 495 | 460-520 | 510-730 |
| Janus Green | 654 | 630-690 | 668-850 |
| Neutral Red | 541 | 520-560 | 555-740 |
| Bismark Brown | 468 | 450-480 | 480-730 |
| Carbol Fuchsin | 550 | 530-570 | 565-720 |
| Brilliant Cresyl Blue | 630 | 610-650 | 645-780 |
| Methylene Blue Chloride | 662 | 640-690 | 674-800 |
| Fluorescein Isothiocyanate | 488 | 460-520 | 510-700 |
| Reichardt's Dye | | | |

In one example, the following Matlab code may be used by processing circuitry to generate data indicative of the age of plastics using RGB data as described herein. This is just one example set of code, but others can be used to achieve the functions described herein. In general, the code takes images using camera 2. RGB extracted from each pixel is used to calculate r/g and g/r ratios. Ratios are used to extra the wavelength for each pixel. Each pixel is separated by wavelength of interest. This data sets are converted back to RGB. Resulting figures break raw image into degraded classes depending on spectral region of interest (e.g., the wavelength areas of interest). % Original sample figure is shown with age

```
% Original sample figure is shown with age
%image data processing
cam = webcam(1);
img = snapshot(cam);
figure(1);
imshow(img);
colorin = img;
rcolor = im2double(colorin(:,:,1));
gcolor = im2double(colorin(:,:,2));
bcolor = im2double(colorin(:,:,3));
%calculate r/G ratio - required to recover color information
rgratio = rcolor./gcolor;%use to represent perceived color
grratio = gcolor./rcolor;
%ci = (rgratio-570.4)./15.63; % from Figure 8, Jon's thesis
%breaking up by wavelength
% 570-575
% 580
% 590
% 600-800
%remove non-numbers
x1 = rgratio;
x1(isinf(x1) | isnan(x1)) = 0;
color = −65.*(x1).\hat{ }−1.252+645;
color(isinf(color)) = 0; %make colors using rg ratio
%break colors up
  for i = 1:768
    for j = 1:1024
      if color(i,j) > 570
        color(i,j) = color(i,j);
      else
        color(i,j) = 0;
      end
    end
  end
  %create data dumps
colorg = zeros(768,1024);
color1 = zeros(768,1024); %570-575
color2 = zeros(768,1024); %576-580
color3 = zeros(768,1024); %581-590
color4 = zeros(768,1024); %591-600
color5 = zeros(768,1024); %601-620
color6 = zeros(768,1024); %621-800
  %breaking up color data
  for i = 1:768
    for j = 1:1024
      if color(i,j) > 525
          if color(i,j) < 570
              colorg(i,j) = color(i,j)
          elseif color(i,j) < 575
              color1(i,j) = color(i,j);
          elseif color(i,j) < 580
              color2(i,j) = color(i,j);
          elseif color(i,j) < 590
              color3(i,j) = color(i,j);
            elseif color(i,j) < 600
              color4(i,j) = color(i,j);
            elseif color(i,j) < 620
              color5(i,j) = color(i,j);
            elseif color(i,j) < 800
              color6(i,j) = color(i,j);
          end
      end
    end
  end
  %now color code for computer
  for i = 1:768
    for j = 1:1024
      colorin = colorg(i,j);
      if colorin > 525
          rgval = wavelength2color(colorin)
          rcoloroutg(i,j) = rgval(1);
          gcoloroutg(i,j) = rgval(2);
      else
          rcoloroutg(i,j) = 0;
          gcoloroutg(i,j) = 0;
      end
        colorin = color1(i,j);
        if colorin > 569
        rgval = wavelength2color(colorin);
        rcolorout1(i,j) = rgval(1);
        gcolorout1(i,j) = rgval(2);
        else
            rcolorout1(i,j) = 0;
            gcolorout1(i,j) = 0;
        end
      %
        colorin2 = color2(i,j);
        if colorin2 > 569
        rgval2 = wavelength2color(colorin2);
        rcolorout2(i,j) = rgval2(1);
        gcolorout2(i,j) = rgval2(2);
        else
            rcolorout2(i,j) = 0;
            gcolorout2(i,j) = 0;
        end
        %
        colorin3 = color3(i,j);
        if colorin3 > 569
        rgval3 = wavelength2color(colorin3);
        rcolorout3(i,j) = rgval3(1);
        gcolorout3(i,j) = rgval3(2);
        else
            rcolorout3(i,j) = 0;
            gcolorout3(i,j) = 0;
        end
        %
        colorin4 = color4(i,j);
        if colorin4 > 569
        rgval4 = wavelength2color(colorin4);
        rcolorout4(i,j) = rgval4(1);
        gcolorout4(i,j) = rgval4(2);
        else
            rcolorout4(i,j) = 0;
            gcolorout4(i,j) = 0;
        end
        %
        colorin5 = color5(i,j);
        if colorin5 > 569
        rgval5 = wavelength2color(colorin5);
        rcolorout5(i,j) = rgval5(1);
        gcolorout5(i,j) = rgval5(2);
        else
            rcolorout5(i,j) = 0;
            gcolorout5(i,j) = 0;
        end
        colorin6 = color6(i,j);
        if colorin6 > 569
        rgval6 = wavelength2color(colorin6);
        rcolorout6(i,j) = rgval6(1);
        gcolorout6(i,j) = rgval6(2);
        else
            rcolorout6(i,j) = 0;
            gcolorout6(i,j) = 0;
        end
    end
  end
  colorgimg = cat(3,255*rcoloroutg, 255*gcoloroutg,zeros(size(rcoloroutg)));
  colorgimg = uint8(colorgimg);
  color1img = cat(3,255*rcolorout1, 255*gcolorout1,zeros(size(rcolorout1)));
  color1img = uint8(color1img);
  color2img = cat(3,255*rcolorout2, 255*gcolorout2,zeros(size(rcolorout2)));
  color2img = uint8(color2img);
  color3img = cat(3,255*rcolorout3, 255*gcolorout3,zeros(size(rcolorout3)));
  color3img = uint8(color3img);
  color4img = cat(3,255*rcolorout4, 255*gcolorout4,zeros(size(rcolorout4)));
  color4img = uint8(color4img);
  color5img = cat(3,255*rcolorout5, 255*gcolorout5,zeros(size(rcolorout5)));
  color5img = uint8(color5img);
  color6img = cat(3,255*rcolorout6, 255*gcolorout6,zeros(size(rcolorout6)));
  color6img = uint8(color6img);
figure(1);clf;
subplot(2,4,1)
  imshow(img)
  subplot(2,4,2)
  imshow(colorgimg)
  title(‘No aging’)
  subplot(2,4,3)
  imshow(color1img)
title(‘0 h/0.2 CI’)
    subplot(2,4,4)
  imshow(color2img)
title(‘4 h/0.35 CI’)
    subplot(2,4,5)
  imshow(color3img)
title(‘8 h/0.5 CI’)
    subplot(2,4,6)
  imshow(color4img)
title(‘12 h/0.65 CI’)
    subplot(2,4,7)
  imshow(color5img)
title(‘24 h/1.0 CI’)
    subplot(2,4,8)
  imshow(color6img)title(‘48 h/2.2 CI’)
```

As described herein, data demonstrates fluorescent dye Nile red as a quantitative technique for monitoring degradation of polymer films. Overall, this tool can be effective at characterizing a wide range of degradation states, included the early stages that are typically hard to characterize with more traditional materials characterization tools. Clear trends have been established between spectral changes in Nile red stained plastics, as monitored by peak ratio fluorescence tracking, and current methods of polymer degradation monitoring, including bulk crystallinity from DSC and carbonyl index from FTIR. Trends observed in stained PE are shown to independent of film thickness and independent of stained film storage times, supporting the longevity and versatility of this technique in application to a variety of plastic morphologies. Additionally, consistent trends are seen between photo- and thermal oxidation degradation methods. Lastly, there is a demonstration of concept when applied to other initially hydrophobic polymers such PP and to commercial plastic samples such as single use plastic bags that contain a variety of different color additives.

Overall, the power of this technique can include monitoring the degradation over the course of plastic degradation is apparent, especially at the earliest stages of oxidation where other current techniques fall short. There is also a flexibility and applicability to this technique that allows for a greater expansion of utilization in many aspects of material characterization and materials research. This technique can be used for a variety of purposes, such as improving industrial recycling ability. This staining strategy and analysis can be a powerful technique for plastic researchers to investigate and track stages of degradation that are currently difficult to monitor with available techniques, as just some examples.

The following examples are described herein:

Example 1. A method comprising: controlling a light source to deliver first light to a plastic material stained with a stain; receiving, by a light detector, second light, the second light comprising at least some of the first light that has interacted with the plastic material stained with the stain; generating, by the light detector, light information representative of the second light; determining, by processing circuitry and based on the light information, an age of the plastic material; and outputting, by the processing circuitry and for display at a display of a computing device, information indicative of the age of the plastic material.

Example 2. The method of example 1, further comprising staining the plastic material with the stain, wherein the stain comprises Nile red.

Example 3. The method of example 2, wherein staining the plastic material comprises staining the plastic material with the stain for a predetermined amount of time.

Example 4. The method of any of examples 1 through 3, wherein the receiving the second light comprises receiving, by the light detector of the computing device, the second light.

Example 5. The method of any of examples 1 through 4, wherein the first light comprises 488 nanometer (nm) laser light.

Example 6. The method of any of examples 1 through 5, further comprising, prior to directing the first light to the plastic material, passing the first light through a dispersion lens.

Example 7. The method of any of examples 1 through 6, further comprising, prior to receiving the second light, passing unfiltered light through a green fluorescent protein (GFP) dichroic filter configured to pass wavelengths from 505 nanometers (nm) to 800 nm.

Example 8. The method of any of examples 1 through 7, further comprising processing the second light by at least: determining pixel intensities for the second light at a plurality of pixels of the light detector; converting the pixel intensities to respective wavelengths of light; and determining a ratio of wavelengths corresponding to green light to wavelengths corresponding to red light.

Example 9. The method of example 8, wherein determining the age of the plastic material comprises determining the age of the plastic material based on the ratio of the wavelengths.

Example 10. The method of any of examples 1 through 9, further comprising outputting, for display, data representative of the light information.

Example 11. The method of any of examples 1 through 10, further comprising displaying, by the display, the age of the plastic material.

Example 12. A system comprising: processing circuitry configured to: control a light source to deliver first light to a plastic material stained with a stain; receive, light information generated by a light detector and representative of second light detected by the light detector, the second light comprising at least some of the first light that has interacted with the plastic material stained with the stain; determine, based on the light information, an age of the plastic material; and output, for display at a display of a computing device, information indicative of the age of the plastic material.

Example 13. The system of example 12, wherein the stain comprises Nile red.

Example 14. The system of any of examples 12 and 13, further comprising the light detector configured to detect the second light and generate the light information.

Example 15. The system of any of examples 12 through 14, wherein the first light comprises 488 nanometer (nm) laser light.

Example 16. The system of any of examples 12 through 15, further comprising: the light source and a dispersion lens, wherein the light source is positioned with respect to the dispersion lens such that, prior to directing the first light to the plastic material, the light source passes the first light through the dispersion lens; and a green fluorescent protein (GFP) dichroic filter configured to pass wavelengths from 505 nanometers (nm) to 800 nm, wherein the second light is passed through the GFP dichroic filter.

Example 17. The system of any of examples 12 through 16, wherein the processing circuitry is further configured to process the second light by at least: determining pixel intensities for the second light at a plurality of pixels of the light detector; converting the pixel intensities to respective wavelengths of light; and determining a ratio of wavelengths corresponding to green light to wavelengths corresponding to red light.

Example 18. The system of example 17, wherein the processing circuitry is configured to determine the age of the plastic material by at least determining the age of the plastic material based on the ratio of the wavelengths.

Example 19. The system of any of examples 12 through 18, further comprising a display configured to output, for display, the information indicative of the age of the plastic material.

Example 20. Computer-readable medium comprising instructions that, when executed, cause processing circuitry to: control a light source to deliver first light to a plastic material stained with a stain; receive, light information generated by a light detector and representative of second light detected by the light detector, the second light comprising at least some of the first light that has interacted with the plastic material stained with the stain; determine, based on the light information, an age of the plastic material; and output, for display at a display of a computing device, information indicative of the age of the plastic material.

Example 21. A computing device comprising: a display; and processing circuitry having access to a memory device, the processing circuitry configured to: generate light information representative of fluorescent light emitted from a combination of Nile red stain and a plastic material; determine, based on the light information, an age of the plastic material from the processing; and control the display to output the determined age of plastic material.

Example 14. A system comprising: a light source configured to direct light on a combination of plastic material and Nile red stain; and a computing device comprising: processing circuitry having access to a memory device, the processing circuitry configured to: generate fluorescent light emitted from the solution of the Nile red and plastic material; process the received fluorescent light; determine an age of the plastic material from the processing; and output the determined age of plastic material and the processed data within a display of the computing device.

The techniques described herein may be implemented in hardware, software, firmware, or any combination thereof. Various features described as modules, units or components may be implemented together in an integrated logic device or separately as discrete but interoperable logic devices or other hardware devices. In some cases, various features of electronic circuitry may be implemented as one or more integrated circuit devices, such as an integrated circuit chip or chipset.

If implemented in hardware, this disclosure may be directed to an apparatus such as a processor or an integrated circuit device, such as an integrated circuit chip or chipset. Alternatively or additionally, if implemented in software or firmware, the techniques may be realized at least in part by a computer-readable data storage medium comprising instructions that, when executed, cause a processor to perform one or more of the methods described above. For example, the computer-readable data storage medium may store such instructions for execution by a processor.

A computer-readable medium may form part of a computer program product, which may include packaging materials. A computer-readable medium may comprise a computer data storage medium such as random access memory (RAM), read-only memory (ROM), non-volatile random access memory (NVRAM), electrically erasable programmable read-only memory (EEPROM), Flash memory, magnetic or optical data storage media, and the like. In some examples, an article of manufacture may comprise one or more computer-readable storage media.

In some examples, the computer-readable storage media may comprise non-transitory media. The term “non-transitory” may indicate that the storage medium is not embodied in a carrier wave or a propagated signal. In certain examples, a non-transitory storage medium may store data that can, over time, change (e.g., in RAM or cache).

The code or instructions may be software and/or firmware executed by processing circuitry including one or more processors, such as one or more digital signal processors (DSPs), general purpose microprocessors, application-specific integrated circuits (ASICs), field-programmable gate arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, functionality described in this disclosure may be provided within software modules or hardware modules.

In addition to or as an alternative to the above, the following examples are described. The features described in any of the following examples may be utilized with any of the other examples described herein.

What is claimed is:

1. A method comprising:
controlling a light source to deliver first light to a plastic material stained with a stain;
receiving, by a light detector, second light, the second light comprising at least some of the first light that has interacted with the plastic material stained with the stain;
generating, by the light detector, light information representative of the second light;
determining, by processing circuitry and based on the light information, an age of the plastic material; and
outputting, by the processing circuitry and for display at a display of a computing device, information indicative of the age of the plastic material.

2. The method of claim 1, further comprising staining the plastic material with the stain, wherein the stain comprises Nile red.

3. The method of claim 2, wherein staining the plastic material comprises staining the plastic material with the stain for a predetermined amount of time.

4. The method of claim 1, wherein the receiving the second light comprises receiving, by the light detector of the computing device, the second light.

5. The method of claim 1, wherein the first light comprises 488 nanometer (nm) laser light.

6. The method of claim 1, further comprising, prior to directing the first light to the plastic material, passing the first light through a dispersion lens.

7. The method of claim 1, further comprising, prior to receiving the second light, passing unfiltered light through a green fluorescent protein (GFP) dichroic filter configured to pass wavelengths from 505 nanometers (nm) to 800 nm.

8. The method of claim 1, further comprising processing the second light by at least:
determining pixel intensities for the second light at a plurality of pixels of the light detector;
converting the pixel intensities to respective wavelengths of light; and
determining a ratio of wavelengths corresponding to green light to wavelengths corresponding to red light.

9. The method of claim 8, wherein determining the age of the plastic material comprises determining the age of the plastic material based on the ratio of the wavelengths.

10. The method of claim 1, further comprising outputting, for display, data representative of the light information.

11. The method of claim 1, further comprising displaying, by the display, the age of the plastic material.

12. A system comprising:
processing circuitry configured to:
control a light source to deliver first light to a plastic material stained with a stain;
receive, light information generated by a light detector and representative of second light detected by the light detector, the second light comprising at least some of the first light that has interacted with the plastic material stained with the stain;
determine, based on the light information, an age of the plastic material; and
output, for display at a display of a computing device, information indicative of the age of the plastic material.

13. The system of claim 12, wherein the stain comprises Nile red.

14. The system of claim 12, further comprising the light detector configured to detect the second light and generate the light information.

15. The system of claim 12, wherein the first light comprises 488 nanometer (nm) laser light.

16. The system of claim 12, further comprising:
the light source and a dispersion lens, wherein the light source is positioned with respect to the dispersion lens such that, prior to directing the first light to the plastic material, the light source passes the first light through the dispersion lens; and
a green fluorescent protein (GFP) dichroic filter configured to pass wavelengths from 505 nanometers (nm) to 800 nm, wherein the second light is passed through the GFP dichroic filter.

17. The system of claim 12, wherein the processing circuitry is further configured to process the second light by at least:
determining pixel intensities for the second light at a plurality of pixels of the light detector;
converting the pixel intensities to respective wavelengths of light; and
determining a ratio of wavelengths corresponding to green light to wavelengths corresponding to red light.

18. The system of claim 17, wherein the processing circuitry is configured to determine the age of the plastic material by at least determining the age of the plastic material based on the ratio of the wavelengths.

19. The system of claim 12, further comprising a display configured to output, for display, the information indicative of the age of the plastic material.

20. Computer-readable medium comprising instructions that, when executed, cause processing circuitry to:
control a light source to deliver first light to a plastic material stained with a stain;
receive, light information generated by a light detector and representative of second light detected by the light detector, the second light comprising at least some of the first light that has interacted with the plastic material stained with the stain;
determine, based on the light information, an age of the plastic material; and
output, for display at a display of a computing device, information indicative of the age of the plastic material.
